(19) United States
(12) Patent Application Publication
(10) Pub. No.: US 2010/0204100 A1
(43) Pub. Date: Aug. 12, 2010

(54) BIOLOGICAL AGENTS ACTIVE IN CENTRAL NERVOUS SYSTEM

(75) Inventors: Roger A. Johns, Reisterstown, MD (US); Feng Tao, Baltimore, MD (US)

Correspondence Address: BANNER & WITCOFF, LTD., 1100 13th STREET, N.W., SUITE 1200, WASHINGTON, DC, 20005-4051, US

(73) Assignee: THE JOHNS HOPKINS UNIVERISTY, Baltimore, MD (US)

(21) Appl. No.: 12/596,330

(22) PCT Filed: Apr. 17, 2008
(86) PCT No.: PCT/US08/60627

§ 371 Date: Dec. 9, 2009

Related U.S. Application Data
(60) Provisional application No. 60/925,325, filed on Apr. 19, 2007.
(60) Provisional application No. 60/925,322, filed on Apr. 19, 2007.

(51) Int. Cl. A61K 38/16; C07K 14/00; A61P 25/00; A61P 23/00

(52) U.S. Cl. 514/12; 530/350

(57) ABSTRACT

Cell-permeant fusion peptides Tat-PDZ can dose-dependently reduce the threshold for anesthesia. PDZ domain-mediated protein interactions at synapses in the central nervous system play an important role in the molecular mechanisms of anesthesia. Moreover, Tat-PDZ cell-permeant fusion peptides are delivered intracellularly into neurons in the central nervous system subsequent to intraperitoneally injection. By in vitro and in vivo binding assays, we found that the Tat-PDZ dose-dependently inhibited the interactions between NMDARs and PSD-95. Furthermore, behavior testing showed that animals given Tat-PDZ exhibited significantly reduced established inflammatory pain behaviors compared to vehicle-treated group. Our results indicate that by disrupting NMDAR/PSD-95 protein interactions, the Tat-PDZ cell-permeable fusion peptides provide a new approach for inflammatory pain therapy.

This application claims the benefit of provisional applications Ser. No. 60/925,322 filed Apr. 19, 2007, and Ser. No. 60/925,325 filed Apr. 19, 2007, the entire contents of which are expressly incorporated herein.

This invention was made using funds from the U.S. government under grants from the National Institutes of Health numbered RO1 GM049111-12, GM049111-13A2, and RO1 NS044219-04. The U.S. government therefore retains certain rights in the invention.

### BACKGROUND OF THE INVENTION

Chronic pain secondary to injury and inflammation is a prevalent problem that can be debilitating to patients. However, many of the currently available pain therapies either are inadequate or cause uncomfortable to deleterious side effects (Eisenberg, et al., 2006; Eisenberg, et al., 2005; Dworkin, et al., 2003; Hansson and Dickenson, 2005; Bertolini, et al., 2002; Laird, et al., 1997; Feldmann, 2002; Reimold, 2003). Several lines of evidence have demonstrated that the activation of N-methyl-D-aspartate receptors (NMDARs) plays an important role in the processing of nociceptive information (Garry, et al., 2000; Mao, et al., 1992; Ren, et al., 1992; Wei, et al., 2001). Postsynaptic density protein-95 (PSD-95), a scaffolding protein, has been identified to attach NMDARs to internal signaling molecules at neuronal synapses of the central nervous system (CNS) (Christopherson, et al., 1999; Kornau, et al., 1995). This function suggests that PSD-95 might be involved in physiological and pathophysiological actions triggered via the activation of NMDARs in the CNS. Therefore, targeting PSD-95 protein represents a potential therapeutic approach for diseases that involve NMDAR signaling.

NMDAR/PSD-95 protein interactions are mediated by a PDZ domain (a term derived from the names of the first three proteins identified to contain the domain: PSD-95, Dlg, and ZO-1). PSD-95 possesses three PDZ domains. The second PDZ domain of PSD-95 (PSD-95 PDZ2) interacts with the seven-amino acid, COOH-terminal domain containing a terminal tSXV motif (where S is serine, X is any amino acid, and V is valine) common to NR2 subunits of NMDARs (Kornau, et al., 1995). The PSD-95 PDZ2 also forms a heterodimeric PDZ-PDZ interaction with the PDZ domain of neuronal nitric oxide synthase (nNOS) (Brenman, et al., 1996b; Brenman, et al., 1996a). The coupling of nNOS to the NMDARs by the PDZ domain of PSD-95 facilitates NMDA activation of nNOS, which is critical to neuronal plasticity, learning, memory, and behavior (Bliss and Collingridge, 1993; Jaffrey and Snyder, 1995; Nelson, et al., 1995)

N-methyl-d-aspartate receptor (NMDAR) activation has been demonstrated to play an important role in the processing of spinal nociceptive information¹⁻⁴ and in the determination of the minimum alveolar anesthetic concentration (MAC) of inhalational anesthetics⁵⁻¹¹. Postsynaptic density protein-95 (PSD-95), a scaffolding protein, has been identified to attach NMDARs to internal signaling molecules at neuronal synapses of the central nervous system (CNS)^{12;13}. This function suggests that PSD-95 might be involved in physiological and pathophysiological actions triggered via the activation of NMDARs in the CNS. NMDAR/PSD-95 protein interactions are mediated by a PDZ domain (a term derived from the names of the first three proteins identified to contain the domain: PSD-95, Dlg, and ZO-1). PSD-95 possesses three PDZ domains. The second PDZ domain of PSD-95 (PSD-95 PDZ2) interacts with the seven-amino acid, COOH-terminal domain containing a terminal tSXV motif (where S is serine, X is any amino acid, and V is valine) common to NR2 subunits of NMDARs¹³. PSD-95 PDZ2 also interacts with the Shaker-type Kv1.4 potassium channel and this interaction regulates the clustering of PSD-95 with the Kv1.4 channel¹⁴.

Our previous studies have shown that the expression of spinal PSD-95 is critical for NMDAR-mediated thermal hyperalgesia (Tao, et al., 2000), and that the knockdown of spinal PSD-95 produced by intrathecal injection of PSD-95 antisense oligodeoxynucleotide delays the onset of neuropathic pain and diminishes the maintenance of pain behaviors (Tao, et al., 2001; Tao, et al., 2003a). In addition, Our previous studies have shown that clinically relevant concentrations of inhalational anesthetics dose-dependently and specifically inhibit the PDZ domain-mediated protein interaction between PSD-95 and NMDARs¹⁵. These inhibitory effects are immediate, potent, and reversible and occur at a hydrophobic peptide-binding groove on the surface of the PSD-95 PDZ2 in a manner relevant to anesthetic action¹⁵. These findings reveal the PDZ domain as a new molecular target for inhalational anesthetics. We have also found that PSD-95 knockdown significantly reduced MAC for isoflurane and attenuated the NMDA-induced increase in isoflurane MAC¹⁶.

There is a need in the art for new ways of treating and preventing hyperalgesia and chronic and acute pain. In addition, there is a need in the art for new and safer ways of rendering patients unconscious or sedation.

### SUMMARY OF THE INVENTION

One aspect of the invention provides a method for relieving acute or chronic pain in a human. An effective amount of a fusion protein which comprises a cell membrane transduction domain of HIV1 Tat and a PDZ domain of a protein selected from the group consisting of PICK1, PSD93 and PSD95, is administered to a subject in need thereof. Acute or chronic pain experienced by the subject is thereby relieved.

Another aspect of the invention provides a method for treating or preventing allodynia or hyperalgesia in a human. An effective amount of a fusion protein which comprises a cell membrane transduction domain of HIV1 Tat and a PDZ domain of a protein selected from the group consisting of PICK1, PSD93 and PSD95, is administered to a subject in need thereof. Allodynia or hyperalgesia experienced by the subject is thereby relieved.

Another aspect of the invention is a method of reducing a threshold for anesthesia in a human. An anesthetic and a fusion protein which comprises a cell membrane transduction domain of HIV1 Tat and a PDZ domain of a protein selected from the group consisting of MUPP1, PSD93 and PSD95, is administered to a subject. The amount of anesthetic administered is less than the amount required in the absence of the agent to achieve a desired anesthetic effect. Nonetheless the desired anesthetic effect is achieved.

The present invention also provides an isolated and purified fusion protein which comprises a cell membrane transduction domain of HIV1 Tat and a PDZ domain of a protein selected from the group consisting of PICK1, MUPP1, PSD95 and PSD93.

Another aspect of the invention is a method of anesthetizing or sedating a human. A fusion protein which comprises a cell membrane transduction domain of HIV1 Tat and a PDZ domain of a protein selected from the group consisting of MUPP1, PSD93 and PSD95, is administered to a subject. The agent thereby renders the subject unconscious or sedated.

Also provided is a composition comprising at least two isolated and purified fusion proteins which each comprise a cell membrane transduction domain of HIV1 Tat and a PDZ domain of a protein selected from the group consisting of PICK1, MUPP1, PSD95 and PSD93.

These and other aspects of the invention provide the art with biologicals for pain management, sedation, and anesthesia which can replace or be used in conjunction with existing chemical agents.

### BRIEF DESCRIPTION OF THE DRAWINGS

FIG. 1A-1C. In vitro and in vivo intracellular delivery of Tat-PSD-95 PDZ2 into mouse spinal cord neurons.

FIG. 1A, After incubation with His-tagged fusion peptides (Tat-PSD-95 PDZ2 or PSD-95 PDZ2 without Tat) for 30 min, Western blotting with anti-His antibody showed that the His-peptide was detected only in the neurons treated with Tat-PSD-95 PDZ2, but not in the neurons treated with PSD-95 PDZ2 or medium alone. Tubulin served as a loading control.

FIG. 1B, Western blot analysis demonstrated that after intraperitoneal injection, Tat-linked fusion peptides (Tat-PSD-95 PDZ2 and mutated Tat-PSD-95 PDZ2) were delivered into lumbar spinal cord of mice; PSD-95 PDZ2 without Tat was not detected in the spinal cord. Tat-PSD-95 PDZ2 was dose-dependently delivered into the spinal cord following systemic administration. 1: PSD-95 PDZ2 without Tat (8 mg/kg); 2: mutated Tat-PSD-95 PDZ2 (8 mg/kg); 3: Tat-PSD-95 PDZ2 (2 mg/kg); 4: Tat-PSD-95 PDZ2 (4 mg/kg); 5: Tat-PSD-95 PDZ2 (8 mg/kg). Tubulin served as a loading control.

FIG. 1C, Immunohistochemical staining demonstrated that only fusion peptide linked to Tat (Tat-PSD-95 PDZ2) was distributed in the spinal cord after intraperitoneal injection. The Tat fusion peptide was accumulated in the cell bodies of the spinal cord (a & b), but PSD-95 PDZ2 was not detected in the spinal cord after systemic administration (c & d). a & b: Tat-PSD-95 PDZ2; c & d: PSD-95 PDZ2. b & d represent high magnification of the outlined areas in a & c, respectively. Scale bars: 50 μm (X 10); 10 μm (X 40). The data shown are representative of three independent experiments.

FIG. 2A-2B. Disruption of NMDAR/PSD-95 protein interactions by Tat-PSD-95 PDZ2.

FIG. 2A, GST pull-down showed that Tat-PSD-95 PDZ2 dose-dependently inhibited the interactions between NMDA receptor NR2B and PSD-95 protein; mutated Tat-PSD-95 PDZ2 had no effect.

FIG. 2B, Co-immunoprecipitation showed that Tat-PSD-95 PDZ2 (8 mg/kg) markedly blocked the interaction between NR2A/2B and PSD-95 and that mutated Tat-PSD-95 PDZ2 (8 mg/kg) had no effect on this interaction compared to the effect of PSD-95 PDZ2 (8 mg/kg). The specificity of the NR2A/2B antibody was verified by preincubation with NR2 peptide. The amount of sample loaded for the input was 10% of that for the immunoprecipitation. IP: immunoprecipitation; IB: immunoblotting. The data shown are representative of three independent experiments.

FIG. 3. Intraperitoneal (i.p.) and intrathecal (i.t.) injection with Tat-PSD-95 PDZ2 significantly inhibited CFA-induced inflammatory pain behaviors in both development and maintenance phases.

FIG. 3A, After i.p. injection, Tat-PSD-95 PDZ2 at 2 mg/kg (n=8), 4 mg/kg (n=8), and 8 mg/kg (n=8) dose-dependently inhibited the CFA-induced decrease of paw withdrawal threshold on the ipsilateral side. *p<0.05 vs. the vehicle-treated group (0 mg/kg; n=6).

FIG. 3B, After i.t. injection, Tat-PSD-95 PDZ2 at 2.5 μg/5 μl (n=8), 5 μg/5 μl (n=8), and 10 μg/5 μl (n=8) dose-dependently inhibited the CFA-induced decrease of paw withdrawal threshold on the ipsilateral side. *p<0.05 vs. the vehicle-treated group (0 μg/5 μl; n=6).

FIG. 4. Table 1. Intraperitoneal injection of Tat peptides had no effect on locomotor function of unanesthetized mice.

FIG. 5A-5B. Disruption of NMDAR/PSD-95 protein interactions by Tat-PSD-95 PDZ2.

FIG. 5A, GST pull-down showed that Tat-PSD-95 PDZ2 dose-dependently inhibited the interactions between NMDA receptor NR2B and PSD-95 protein; mutated Tat-PSD-95 PDZ2 had no effect.

FIG. 5B, Co-immunoprecipitation showed that Tat-PSD-95 PDZ2 (8 mg/kg) markedly blocked the interaction between NR2A/2B and PSD-95 and that mutated Tat-PSD-95 PDZ2 (8 mg/kg) had no effect on this interaction compared to the effect of PSD-95 PDZ2 (8 mg/kg). The specificity of the NR2A/2B antibody was verified by preincubation with NR2 peptide. The amount of sample loaded for the input was 10% of that for the immunoprecipitation. IP: immunoprecipitation; IB: immunoblotting.

FIG. 6A-6B. Intrathecal injection with Tat-PSD-95 PDZ2 significantly inhibited CFA-induced inflammatory pain behaviors in both development and maintenance phases.

FIG. 7.A, Tat-PSD-95 PDZ2 dose-dependently inhibited the CFA-induced decrease of paw withdrawal threshold on the ipsilateral side, but mutated Tat-PSD-95 PDZ2 or PSD-95 PDZ2 had no effect. *p<0.05 VS. the vehicle-treated group.

FIG. 7.B, On the contralateral side, these peptides did not significantly influence paw withdrawal threshold after intrathecal injections.

FIG. 7A-7B. Intraperitoneal injection with Tat-PSD-95 PDZ2 had no effect on the baseline behavior and locomotor function of mice.

FIG. 8A, After intraperitoneal injection, these fusion peptides including Tat-PSD-95 PDZ2 had no significant effect on the baseline paw withdrawal threshold of the mice.

FIG. 8B, After intraperitoneal injection, these fusion peptides including Tat-PSD-95 PDZ2 did not show any effect on the tests of locomotor function.

### DETAILED DESCRIPTION OF THE INVENTION

It is a discovery of the present inventors that fusion proteins comprising the HIV1 TAT protein cellular permeability domain and a PDZ domain of certain proteins can provide effective inhibition of pain, anesthetic and sedative effects, and reduction of anesthetic thresholds. The PDZ domains are obtained from binding partners of cellular receptors involved in neuronal function, such as the AMPA, NMDA, and GABA receptors and kv1.4 channels. Such binding partners include MUPP1, PICK1, PSD93, and PSD95. Other similar binding partners to cellular receptors involved in neuronal function that have PDZ interactions may also be used. Each of these proteins is known in the art. Exemplary human sequences are provided in the sequence listing portion of this application. Proteins that differ by up to 1, 2, 3, 5, 7, 10, 12, or 15% of their amino acid residues can be used similarly, provided that PDZ binding interactions are maintained. Variants of the sequences disclosed may be polymorphisms that occur in the population or changes that are introduced synthetically.

PDZ domains from any protein can be used in the fusion proteins of the invention. These include AF-6, AIE-75/harmonin, MAGI-2, MAGI-3, CASK, Delphilin, ERBIN, GIPC, GOPC/PIST, IKEPP, PTPL1, PTPase-MEG1, MP55, Shank1, Shank2, TIP-1, Veli-1, Veli-2, Veli-3, ZO-1, SAP102, SAP97, MUPP1, NHERF-1, NHERF-2, PDZ-RhoGEF, PDZK1, PICK1, PSD-93, PSD-95, alpha-1-syntrophin, beta-2-syntrophin, gamma-1-syntrophin, gamma-2-syntrophin, hDlt, p55, and PTP-H1.

Fusion proteins may comprise additional sequences, such as linkers, histidine tags, and/or detectable labels. Any moiety which can be useful may be added. These may facilitate efficient synthesis, purification, or tracking within the body when administered. Any suitable protein modification as is known in the art can be used. The modification may be one that can be synthesized as part of the protein within host cells or one that is added chemically after synthesis of the fusion protein.

Some proteins contain multiple PDZ domains. Any can be used, although they may not be equally potent. One can make fusion proteins that contain multiple PDZ domains, from the same or different proteins. One can make mixtures of fusion proteins, each having a different PDZ domain. Such mixtures may comprise two, three, four, or more individual fusion proteins. They PDZ domains may interact with the same or a different cellular target. Combinations of PDZ domains (in one or more fusion protein) may inhibit a single cellular target more potently. Combinations of PDZ domains (in one or more fusion protein) may inhibit different targets, thus providing greater pain relief, sedation, or anesthetic effect.

The effective amount of a fusion protein to be used may depend on the subject to be treated and the effect sought. Thus a large subject may require a higher dosage to achieve the same level of effect as would be required for a smaller subject. A severe pain my require a higher dosage than a milder pain. Rendering a subject unconscious may require a higher dosage than sedation. The potency of the fusion proteins may also affect the precise effective amount. The mode of administration may also affect the dose, with compartmental or direct administration to an affected site requiring a lower dosage than systemic delivery.

Agents according to the present invention can be administered any way known in the art which is convenient and efficient for the particular agent and the application. The agent can be administered intrathecally, per os, intraperitoneally, by inhalation, or intravenously. However, other means can be used as appropriate, including subdermal, subcutaneous, rectal, subarachnoid, caudal, epidural, and intramuscular administrations. Anesthetics and sedatives used in the methods of the present invention can also be administered by any of these same means. Standard anesthetics which may be used in conjunction with the biologicals disclosed herein include inhalational anesthetics, such as halothane, isoflurane, desflurane, xenon, and sevoflurane.

Particular vehicles which are suitable for intrathecal or inhalational therapy can be advantageously used. The formulations can be in liquid or vapor form. They can be vaporized by bubbling a gas through them. Preferably the formulations of the invention will be manufactured under regulatory-approved conditions for administration to humans. Requirements for such formulations may optionally include sterility and freedom from pyrogens.

Fusion proteins can be administered to patients in need of anesthesia, those in need of relief from chronic or acute pain, and those who experience hyperalgesia or are at risk of developing hyperalgesia, and those who experience allodynia. Such patients include those whose pain is mechanical, thermal, neuropathic, or inflammatory in origin. In addition, the fusion proteins can be used to sedate or anesthetize patients, in all situations where this may be needed, including but not limited to surgery, shock, parturition, and trauma.

As an alternative means of treating human subjects, DNA constructs encoding the fusion proteins can be delivered. The DNA constructs may be viral or non-viral vectors as are known in the art. The naturally occurring coding sequences for the portions of the fusion proteins can be used, or other coding sequences which are designed to encode the same amino acids. Liposomes can be used as can DNA-protein complexes and biopolymer complexes. Viruses such as adenovirus, herpes virus, adeno-associated virus, retroviruses, such as lentiviruses, poxyiridae, baculovirus, vaccinia, or Epstein-Barr virus can be used. Expression of the fusion protein may be regulated or constitutive. Expression may be regulated by an internal or external stimulus. Expression may be tissue specific.

This examples below show that intraperitoneally injected fusion peptide Tat-PSD-95 PDZ2 can be delivered into the spinal cord and dose-dependently disrupts the protein-protein interactions between NMDAR NR2 subunits and PSD-95. This peptide significantly inhibits inflammatory sensitization of the behavioral response induced by intraplantar injection of CFA. These results suggest that PDZ domain-mediated protein interactions at spinal synapses might play an important role in the molecular mechanisms of inflammatory pain behaviors.

PTD-mediated in vivo delivery of biologically active peptides represents a novel and promising strategy to treat CNS diseases. Although the exact mechanism of transduction across the cellular membrane is currently unknown, the first step of the process appears to involve a charge-charge interaction of the basic PTD with acidic motifs on the cellular membrane. It has been demonstrated that fusion peptides containing the PTD sequence derived from HIV Tat protein can be transduced into the CNS after systemic administration (Denicourt and Dowdy, 2003). Previous work also has shown that the PTD can be used to efficiently transduce a biologically active neuroprotectant in experimental cerebral ischemia (Cao, et al., 2002). In our study, Tat-PSD-95 PDZ2 (but not PSD-95 PDZ2 without Tat) was successfully transduced into cultured spinal neurons. After intraperitoneal injection, Tat-PSD-95 PDZ2 and mutated Tat-PSD-95 PDZ2 were detected in lumbar spinal cord and other CNS areas (such as, cerebral cortex and hippocampus, data not shown), but PSD-95 PDZ2 without Tat was not delivered into these tissues. These results support the conclusion that a wide variety of cargo, including peptides and full-length proteins, can be delivered into cells when linked to the PTD sequence (Wadia and Dowdy, 2002).

Both the NMDAR subunit NR2B and PSD-95 are highly enriched in the postsynaptic density fraction from the spinal cord (Tao, et al., 2000; Boyce, et al., 1999; Luque, et al., 1994; Garry, et al., 2003) and brain (Moon, et al., 1994; Cho, et al., 1992). Specifically, PSD-95 and NMDARs colocalize at putative synapses in hippocampal pyramidal cells (Kornau, et al., 1995). PSD-95 is distributed mainly in lamina I and outer lamina II of the superficial dorsal horn of the spinal cord (Tao, et al., 2000; Garry, et al., 2003). The expression of the postsynaptic NMDAR subunit NR2B is also limited in laminae I and II of the spinal dorsal horn (Boyce, et al., 1999; Luque, et al., 1994). The interactions between NMDAR NR2 subunits and PSD-95 are mediated by the second PDZ domain of PSD-95 protein (Kornau, et al., 1995). Thus, we hypothesized that competition with a peptide consisting of PSD-95 PDZ2 could disrupt this PDZ domain-mediated protein interactions. Our current results support this hypothesis. By in vitro and in vivo binding assays, we show here that fusion peptide Tat-PSD-95 PDZ2 dose-dependently suppresses the NMDAR/PSD-95 protein interactions. However, mutation of three critical amino acids (K165T, L170R, and H182L) of the PDZ2 domain in the fusion peptide eliminated its ability to affect the interaction.

PDZ domain-mediated protein interactions play a central role in organizing signaling complexes around synaptic receptors for efficient signal transduction. At excitatory synapses of central neurons, ionotropic glutamate receptors are organized into multiprotein signaling complexes within the postsynaptic density (Sheng, 2001). PSD-95 is a prominent organizing protein (Kornau, et al., 1995) that couples the NMDARs to intracellular proteins and signaling enzymes (Brenman, et al., 1996a). Through its second PDZ domain, PSD-95 binds to the COOH-terminus tSXV motif of NMDAR NR2 subunits as well as nNOS (Brenman, et al., 1996a; Kornau, et al., 1995). Therefore, targeting PSD-95 protein and PDZ domain-mediated PSD-95 protein interactions with NMDARs represent potential therapeutic approaches for diseases that involve the dysfunction of NMDA receptors. It has already been shown that disrupting NMDAR/PSD-95 protein interactions reduces focal ischemic brain damage in a stroke model (Aarts, et al., 2002). Also, our previous studies have demonstrated that the expression of spinal PSD-95 is critical for NMDA receptor-mediated hyperalgesia (Tao, et al., 2000), and that the deficiency of spinal PSD-95 inhibits spinal nerve injury-induced pain behavioral responses in both development and maintenance phases (Tao, et al., 2001; Tao, et al., 2003a). Our current data provide additional in vivo evidence to support the novel concept that PDZ domain-mediated protein interactions between NMDARs and PSD-95 is a critical mechanism by which inflammatory sensitization of behavioral response is regulated.

The examples below demonstrate that by disrupting PDZ domain-mediated

NMDAR/PSD-95 protein interactions, the cell-permeable fusion peptide Tat-PSD-95 PDZ2 dose-dependently inhibits CFA-induced established inflammatory pain behaviors. These results provide a novel insight into the molecular mechanisms that underlie the established inflammatory pain states and a new approach for inflammatory pain therapy.

Results from our present studies indicate that intraperitoneally injected fusion peptide Tat-PSD-95 PDZ2 (1) can be delivered into the CNS; (2) dose-dependently disrupts the protein-protein interactions between NMDAR NR2 subunits and PSD-95; and (3) significantly reduces halothane MAC and RREC50. These results suggest that PDZ domain-mediated protein interactions at synapses in the CNS might play an important role in the molecular mechanisms of halothane anesthesia.

PTD-mediated in vivo delivery of biologically active peptides represents a novel and promising strategy to treat CNS diseases. Although the exact mechanism of transduction across the cellular membrane is currently unknown, the first step of the transduction appears to involve a charge-charge interaction of the basic PTD with acidic motifs on the cellular membrane. It has been demonstrated that fusion peptides containing the PTD sequence derived from human immunodeficiency virus Tat protein can be transduced into the CNS after systemic administration²⁴. In our current study, we found that after intraperitoneal injection, Tat-PSD-95 PDZ2 and mutated Tat-PSD-95 PDZ2 were detected in cerebral cortex, hippocampus, and lumbar spinal cord of mice, but PSD-95 PDZ2 lacking Tat was not seen in these tissues. These results support the conclusion that a wide variety of cargo, including peptides and full-length proteins, can be delivered into cells when linked to the PTD sequence²⁵.

The interactions between NMDAR NR2 subunits and PSD-95 are mediated by the second PDZ domain of PSD-95 protein¹³. The Shaker-type potassium channel, Kv1.4, also binds to the PSD-95 PDZ2¹⁴. Thus, we hypothesized that competition with a peptide consisting of PSD-95 PDZ2 could disrupt this PDZ domain-mediated protein interaction. Our current results support this hypothesis. By in vivo binding assay, we show here that fusion peptide Tat-PSD-95 PDZ2 dose-dependently suppresses the NMDAR/PSD-95 protein interaction. However, mutation of three critical amino acids (K165T, L170R and H182L) of the PDZ2 domain in the fusion peptide eliminated its ability to affect the interaction. The mutated Tat-PSD-95 PDZ2 and PSD-95 PDZ2 without Tat served as controls for Tat-PSD-95 PDZ2 in our studies.

Inhalational anesthetics have been in widespread use in modern surgical procedures, but their molecular mechanisms remain poorly understood. PDZ domain-mediated protein interactions play a central role in organizing signaling complexes around synaptic receptors for efficient signal transduction. Our previous studies have demonstrated that halothane dose-dependently and reversibly inhibits PSD-95 PDZ domain-mediated protein interactions, and that the halothane binding site on PSD-95 PDZ2 completely overlaps with the binding pocket of PSD-95 for NMDAR NR2 subunits¹⁵, suggesting a new concept that affecting PDZ domain-mediated protein interactions at synapses in the CNS might be one of molecular mechanisms by which the general anesthetic state is achieved. By knocking down PSD-95 expression in the spinal cord, we have shown that the deficiency of spinal PSD-95 reduced isoflurane MAC in rats¹⁶. In the present study, we found that fusion peptide Tat-PSD-95 PDZ2, but not mutated Tat-PSD-95 PDZ2 or PSD-95 PDZ2, dose-dependently reduced halothane MAC and RREC50 in mice by disrupting the PDZ domain-mediated protein interactions. These results provide in vivo evidence to support this concept. On the other hand, a key concern with inhalational anesthetics is the narrow relationship between the therapeutic and toxic doses. This concern has negative impact on clinical administration of the inhalational anesthetics. Tat-PSD-95 PDZ2, a novel agent, markedly reduces the amount of inhalational anesthetics needed to induce anesthesia, thereby reducing the dose-dependent toxic side effects of the inhalational anesthetics.

The examples below demonstrate that by disrupting PDZ domain-mediated protein interactions, intraperitoneal injection of cell-permeable fusion peptide Tat-PSD-95 PDZ2 dose-dependently reduces the threshold for halothane anesthesia. These results provide a novel insight into the molecular mechanisms that underlie the inhalational anesthetic state and a new target for development of anesthetics.

### EXAMPLES

### Example 1

### Materials and Methods

Animal Preparation. Male C57B1/6J mice 8-10 weeks old were obtained from Jackson Laboratories (Bar Harbor, Mass.) and acclimated in our animal facility for a minimum of 1 week prior to use in experiments. Mice were housed under standard conditions with a 12-h light/dark cycle and allowed food and water ad libitum. All animal experiments were carried out with the approval of the Animal Care and Use Committee at Johns Hopkins University, and adhered to the guidelines of the Committee for Research and Ethical Issues of IASP and the National Institutes of Health guide for the Care and Use of Laboratory Animals (National Institutes of Health Publications No. 8023, revised 1978). All efforts were made to minimize animal suffering, to reduce the number of animals used, and to utilize alternatives to in vivo techniques, if available.

Construction and Purification of Tat Fusion Peptide. The cDNA encoding the second PDZ domain of PSD-95 was prepared in our laboratory as described previously (Fang, et al., 2003). Here, we used sub-cloning to construct a Tat-PSD-95 PDZ2 plasmid by inserting PSD-95 PDZ2 cDNA into the pTAT-HA expression vector, which contains an amino-terminal, in-frame, 11-amino-acid, minimal transduction domain (residues 47-57 of HIV Tat) termed Tat (Becker-Hapak, et al., 2001). Two control plasmids were also constructed: mutated Tat-PSD-95 PDZ2, in which three sites critical for interactions between NMDARs and PSD-95 were mutated (K 165T, L 170 μl and H182L), and PSD-95 PDZ2, which contained the same sequences as Tat-PSD-95 PDZ2 but without Tat PTD. To produce the fusion peptides, these plasmids were transformed into Escherichia coli BL21 cells, and protein expression was induced by 0.5 mM isopropylthiogalactoside at 37° C. for 4 h. The fusion peptides were purified using nickel-nitrilotriacetic acid agarose (Qiagen, Valencia, Calif.) according to a standard 6× histidine (His)-tagged protein purification protocol. The resulting fusion peptides were dialyzed twice against phosphate-buffered saline (PBS). The purified peptides were verified by Coomassie blue staining and Western blot analysis and then stored in 10% glycerol/PBS at −80° C. until use.

In Vitro Delivery of Tat Fusion Peptide in Cultured Spinal Neurons. The intracellular delivery of Tat fusion peptide was assessed by Western blot analysis 30 min after application of 10 μM Tat-PSD-95 PDZ2 to cultured spinal neurons. The same dose of PSD-95 PDZ2 without Tat served as a control. Spinal cord neuronal cultures were prepared as previously described (O'Brien, et al., 1997) with minor modification. In brief, embryonic day 14 mouse spinal cord was digested for 45 min at 34° C. Cells were gently dissociated with a 5-ml pipette, filtered through a 70-μm filter, and centrifuged through a solution of 1% soybean trypsin inhibitor and 1% bovine serum albumin at 80×g for 10 min. High-density cultures were plated at 2 million cells/60-mm dish. Cell lysates from high-density cultures were solubilized in 1% Triton X-100/0.2% sodium dodecyl sulfate (SDS) and subjected to SDS-polyacrylamide gel electrophoresis (SDS-PAGE). Proteins were immunoblotted with monoclonal anti-His antibody (Sigma) and visualized with enhanced chemiluminescence (Amersham Biosciences, Piscataway, N.J.).

In Vivo Systemic Administration and Spinal Cord Distribution of Tat Fusion Peptides. Western blot analysis and immunohistochemistry were used to detect the spinal cord distribution of Tat fusion peptides after systemic administration. The purified fusion peptides at the indicated amounts were injected into mice intraperitoneally in 300 μl of PBS and 10% glycerol, as previously described (Cao, et al., 2002). The mice, assigned randomly to the experimental groups of 6-8, were given Tat-PSD-95 PDZ2 or control peptide (mutated Tat-PSD-95 PDZ2 or PSD-95 PDZ2 without Tat PTD) 4 h before sample collection. For Western blotting, lumbar spinal cord tissues were harvested, homogenized according to standard procedures, and centrifuged at 700×g for 15 min at 4° C. The extracted proteins were resolved by SDS-PAGE, electro-transferred to nitrocellulose membranes, and then immunoblotted with above-mentioned monoclonal anti-His antibody. For immunohistochemistry, sections from the spinal lumbar enlargement segments were fixed in 4% paraformaldehyde (in 0.1 M phosphate buffer, pH 7.4) for 10 min, and then incubated with the monoclonal anti-His antibody. Thereafter, the sections were rinsed with 0.01 M PBS, incubated with fluorescein isothiocyanate-conjugated anti-mouse IgG (1:80; Jackson ImmunoResearch Laboratories) for 1 h at 37° C., and rinsed again with PBS. The sections were imaged with confocal laser scanning microscopy at an excitation wavelength of 488 nm for fluorescein isothiocyanate.

Intrathecal Injection of Tat Fusion Peptides. Intrathecal injection was performed in unanesthetized mice as previously described (Hylden and Wilcox, 1980; Tao, et al., 2003b; Tao, et al., 2004). In brief, the mouse was held firmly by the pelvic girdle in one hand, while a 10-μl Luer tip syringe with a 30 gauge 0.5-inch needle was held in the other hand at an angle of about 20° above the vertebral column. The needle was inserted into the tissue to one side of the L5 or L6 spinous process so that it slipped into the groove between the spinous and transverse processes. The needle was then moved carefully forward to the intervertebral space as the angle of the syringe was decreased to about 10°. A tail flick indicated that the tip of the needle was inserted into the subarachinoid space. The injection volume was 5 μl. The mice, assigned randomly to the experimental groups of 6-8, were given intrathecally Tat-PSD-95 PDZ2 or control peptide (mutated Tat-PSD-95 PDZ2 or PSD-95 PDZ2 without Tat PTD) 30 min before behavioral testing.

In Vitro and In Vivo Binding Assays. Glutathione S-transferase (GST) and GST fusion peptide GST-PSD-95 PDZ1,2 were prepared with glutathione-agarose as an affinity resin (Fang, et al., 2003). Membrane-bound proteins from the spinal lumbar enlargement segments were extracted as described previously (Tao, et al., 2003a). For in vitro binding experiments (GST pull-down), the solubilized membrane fraction and GST fusion peptide were first preincubated with different concentrations of Tat-PSD-95 PDZ2 at room temperature for 30 min. Then the membrane fraction was mixed with the GST fusion peptide at room temperature for 1 h. The resin was washed five times with washing buffer (PBS plus 500 mM NaCl and 0.1% Triton X-100) and then boiled in 1×SDS-PAGE sample buffer to elute the bound proteins. After being separated by electrophoresis, the proteins were detected by immunoblotting with anti-GST antibody (Santa Cruz Biotechnology, Santa Cruz, Calif.) and anti-NR2B antibody (Upstate Biotechnology, Lake Placid, N.Y.). For in vivo binding experiments (co-immunoprecipitation), 5 μg of the affinity-purified rabbit NR2A/2B antibody (Chemicon, Temecula, Calif.) was incubated with 100 μl of protein A-Sepharose slurry for 1 h, and the complex was spun down at 2000 rpm for 4 min. The solubilized membrane fraction (500 μg) from the different groups of treated mice as mentioned-above then was added to the Sepharose beads, and the mixture was incubated for 2-3 h at 4° C. The mixture was washed once with 1% Triton X-100 in immunoprecipitation buffer [containing (in mM): 137 sodium chloride, 2.7 potassium chloride, 4.3 disodium hydrogen phosphate, 1.4 potassium dihydrogen phosphate, 5 ethylene glycol tetraacetic acid, 1 sodium vanadate, 10 sodium pyrophosphate, 50 sodium fluoride, 0.1 phenylmethylsulfonyl fluoride, and 20 U/ml Trasylol], twice with 1% Triton X-100 in immunoprecipitation buffer plus 300 mM sodium chloride, and three times with immunoprecipitation buffer. The proteins were separated by SDS-PAGE and detected by NR2A/2B or PSD-95 antibody (Upstate Biotechnology, Lake Placid, N.Y.). As a positive control (input), 50 μg of the solubilized membrane fraction was loaded onto the gel.

Behavioral Analysis. Mechanical sensitivity of the mice was measured with von Frey filaments (Stoelting, Wood Dale, Ill.) as described previously (Yang and Gereau, 2003). Mice were placed in Plexiglas testing boxes with a 1×1 cm² wire-mesh grid floor and habituated for 3 h before experiments. Each von Frey filament was applied to the mouse hind paw in ascending order until it bent at approximately 30° for about 3 s. The smallest filament that evoked a paw withdrawal response was taken as the mechanical threshold (paw withdrawal threshold). Similar sites were selected for measuring mechanical thresholds in all tested animals, and the thresholds were measured at approximately the same site throughout the experiment for each individual animal. Two to three measurements were made before CFA or fusion peptides injection, and the average was calculated as the baseline. Established inflammatory pain behaviors were induced by intraplantar injection of CFA solution (20 μl, 1 mg/ml). To observe the effect of Tat-PSD-95 PDZ2 on the development of CFA-induced established inflammatory pain, the mice were given intraperitoneally Tat-PSD-95 PDZ2 or control peptide (mutated Tat-PSD-95 PDZ2 or PSD-95 PDZ2 without Tat) 2 h before CFA injection or intrathecally these peptides 1.5 h after CFA injection (development protocol); on the other hand, to observe the effect of Tat-PSD-95 PDZ2 on the maintenance of CFA-induced established inflammatory pain, the mice were given intraperitoneally these peptides 20 h after CFA injection or intrathecally these peptides 23.5 h after CFA injection (maintenance protocol). Behavioral measurements were conducted 2 h after CFA injection for the “development protocol” and 24 h after CFA injection for the “maintenance protocol”. The effect of Tat fusion peptide on CFA-induced established inflammatory pain behaviors was expressed as % of baseline.

Tests for Locomotor Function. Four hours after intraperitoneal injection of Tat fusion peptides, their effects on locomotor function were examined with the following tests as described previously (Coderre and Van, 1,1994; Tao, et al., 2003a). 1) Placing reflex: The mouse was held with hind limbs slightly lower than forelimbs and the dorsal surface of the hind paws was brought into contact with the edge of a table. The experimenter recorded whether the mouse placed its hind paws on the table surface reflexively; 2) Grasping reflex: The mouse was placed on a wire grid and the experimenter recorded whether the hind paws grasped the wire on contact; and 3) Righting reflex: The mouse was placed on its back on a flat surface and the experimenter noted whether it immediately assumed the normal upright position. Scores for these reflexes were based on counts of each normal reflex exhibited in six trials.

Statistical Analysis. Data are expressed as mean±SEM and statistically analyzed with one-way ANOVA followed by Student-Newman-Keuls method. Paired student's t-test was used to compare the difference between pre-injection and post-injection. Statistical significance was set at p<0.05.

### Animal Preparation

Male C57B1/6J mice (8-10 weeks) were obtained from Jackson Laboratories (Bar Harbor, Mass.) and acclimated in our animal facility for a minimum of 1 week prior to use in experiments. The mice were housed under standard conditions with a 12-h light/dark cycle and allowed food and water ad libitum. All animal experiments were carried out with the approval of the Animal Care and Use Committee at Johns Hopkins University and were consistent with the National Institutes of Health Guide for the Care and Use of Laboratory Animals. All efforts were made to minimize the number of animals used and their suffering. The animal assignment was blinded to the observer for all of in vivo testing including MAC measurement, RREC50 determination, and locomotor function test.

### Construction and Purification of Tat Fusion Peptides

The cDNA encoding the PSD-95 PDZ2 was prepared in our laboratory as described previously¹⁵. Here, we used sub-cloning to construct a Tat-PSD-95 PDZ2 plasmid by inserting PSD-95 PDZ2 cDNA into the pTAT-HA expression vector, which contains an amino-terminal, in-frame, 11-amino-acid, minimal transduction domain (residues 47-57 of human immunodeficiency virus Tat protein) termed Tat¹⁷. Two control plasmids were also constructed: mutated Tat-PSD-95 PDZ2, in which three sites critical for interactions between NMDARs and PSD-95 were mutated (K165T, L170R and H182L), and PSD-95 PDZ2, which contained the same sequences as Tat-PSD-95 PDZ2 but without Tat PTD. To produce the fusion peptides, these plasmids were transformed into Escherichia coli BL21 cells, and protein expression was induced by 0.5 mM isopropylthiogalactoside at 37° C. for 4 h. The fusion peptides were purified using Ni-NTA agarose (Qiagen, Valencia, Calif.) according to a standard 6× histidine-tagged protein purification protocol. The resulting fusion peptides were dialyzed twice against phosphate-buffered saline. The purified peptides were verified by Coomassie blue staining and Western blot analysis and then stored in 10% glycerol/phosphate-buffered saline at −80° C. until use.

### In Vivo Administration of Tat Fusion Peptides

The purified fusion peptides at the indicated amounts were injected intraperitoneally into mice in 300 μl of phosphate-buffered saline and 10% glycerol. The mice were given Tat-PSD-95 PDZ2 or control peptide (mutated Tat-PSD-95 PDZ2 or PSD-95 PDZ2 without Tat) 4 h before MAC measurement and righting reflex testing. All the animals were assigned randomly to experimental groups consisting of 6-8 animals each. Western blot analysis was then used to verify the CNS delivery of these fusion peptides after intraperitoneal injection.

### Western Blot Analysis

Cerebral cortex, hippocampus, and lumbar spinal cord were harvested 4 h after intraperitoneal injection of the fusion peptides. Total proteins from these tissues were extracted. In brief, the tissues were removed and homogenized in homogenization buffer¹⁸ (10 mM Tris-HCl, 5 mM MgCl₂, 2 mM EGTA, 1 mM phenylmethylsulfonyl fluoride, 11′-M leupeptin, 2 μM pepstatin A, and 320 mM sucrose, pH 7.4). The crude homogenates were centrifuged at 700×g for 15 mM at 4° C. The pellets were rehomogenized and spun again at 700×g, and the supernatants were combined and diluted in resuspension buffer¹⁸ (10 mM Tris-HCl, 5 mM MgCl₂, 2 mM EGTA, 1 mM phenylmethylsulfonyl fluoride, 1 μM leupeptin, 2 μM pepstatin A, and 250 mM sucrose, pH 7.4). Next, the protein extracts were resolved by sodium dodecyl sulfate-polyacrylamide gel electrophoresis, electrotransferred to nitrocellulose membranes, and then immunoblotted with monoclonal anti-His antibody (Sigma, St. Louis, Mo.) diluted (1:1,000) in blocking solution containing 3% nonfat dry milk and 0.1% Tween-20 in Tris-HCl-buffered saline for 1 h at room temperature. After extensive washing, the membranes were incubated with horseradish peroxidase-conjugated anti-mouse immunoglobulin (Bio-Rad Laboratories, Hercules, Calif.) at a dilution of 1:3,000 for another 1 h. Specific proteins were detected by enhanced chemiluminescence (Amersham, Piscataway, N.J.). Tubulin served as a loading control and cerebral cortex was used for its detection.

### In Vivo Binding Assay: Co-Immunoprecipitation

5 μg of the affinity-purified rabbit NR2A/2B antibody (Chemicon, Temecula, Calif.) was incubated with 100 μl of protein A-Sepharose slurry for 1 h, and the complex was spun down at 2000 rpm for 4 min. The solubilized membrane fraction (500 μg) from the different groups of treated mice as mentioned-above then was added to the Sepharose beads, and the mixture was incubated for 2˜3 h at 4° C. The mixture was washed once with 1% Triton X-100 in immunoprecipitation buffer¹⁹ [containing (in mM): 137 NaCl, 2.7 KCl, 4.3 Na₂HPO₄, 1.4 KH₂PO₄, 5 EGTA, 1 sodium vanadate, 10 sodium pyrophosphate, 50 NaF, and 0.1 phenylmethylsulfonyl fluoride, and 20 U/ml Trasylol], twice with 1% Triton X-100 in immunoprecipitation buffer plus 300 mM NaCl, and three times with immunoprecipitation buffer. The proteins were separated by sodium dodecyl sulfate-polyacrylamide gel electrophoresis and detected by NR2A/2B or PSD-95 antibody (Upstate, Lake Placid, N.Y.). As a positive control (input), 50 μg of the solubilized membrane fraction was loaded onto the gel. The NR2A/2B antibody was preincubated with excess NR2 peptide (100 μg/ml) to verify its specificity.

### Measurement of Halothane MAC

Measurement of halothane MAC value was carried out as previously described with minor modification²⁰⁻²². Mice were placed in individual Plexiglas chambers 3 h after the injection of the fusion peptides. Each chamber was fitted with a rubber stopper at one end through which the mouse's tail and a rectal temperature probe protruded. Groups of four mice were given halothane in oxygen (4 l/min total gas flow). A gas sample was continuously drawn, and the anesthetic concentration was measured with an agent analyzer (Ohmeda 5250 RGM, Louisville, Colo.). A rectal temperature probe was inserted under light general anesthesia, and temperature was kept at 36˜38° C. with heat lamps throughout the experiment. Mice initially breathed approximately 1.5% halothane for 60 min. Next, a 15 cm hemostatic forceps was applied to the tail for 1 min, and the mice were observed for movement in response to the stimulation. In each case, the tail was stimulated proximal to the previous test site. Only the middle third of the tail was used for tail-clamping. The concentration of the anesthetic agent at which the mouse exhibited motor activity (gross movements of the head, extremities, and/or body) was considered one that permitted a positive motor response. The anesthetic concentration was increased (or decreased) in steps of 0.1% until the positive response disappeared (or vice versa), with 10 min for equilibration allowed after each change of anesthetic concentration. MAC is defined as the concentration midway between the highest concentration that permitted movement in response to the stimulus and the lowest concentration that prevented movement.

### Determination of Halothane RREC50

Following the measurement of MAC, the halothane concentration was halved for 10 min and the animal turned on its back to test the righting reflex defined as a return onto all four paws within 1 min²⁰⁻²². The halothane concentration was reduced by 0.1% for 10 min if the animal failed to right itself and the righting reflex subsequently re-tested. RREC50 was calculated for each mouse as the mean value of the anesthetic concentrations that just permitted and just prevented the righting reflex.

### Tests for Locomotor Function

The effects of Tat fusion peptides on locomotor function were examined 4 h after intraperitoneal injection. The following tests were performed as described previously²³. 1) Placing reflex: The mouse was held with hind limbs slightly lower than forelimbs, and the dorsal surface of the hind paws was brought into contact with the edge of a table. The experimenter recorded whether the mouse placed its hind paws on the table surface reflexively; 2) Grasping reflex: The mouse was placed on a wire grid, and the experimenter recorded whether the hind paws grasped the wire on contact. Scores for these reflexes were based on counts of each normal reflex exhibited in six trials.

### Statistical Analysis

Data are expressed as mean±S.E.M. and statistically analyzed with one-way analysis of variance followed by Student-Newman-Keuls method. Statistical significance was set at p<0.05. Statistical analysis was conducted using SigmaStat 2.0 software.

### Example 2

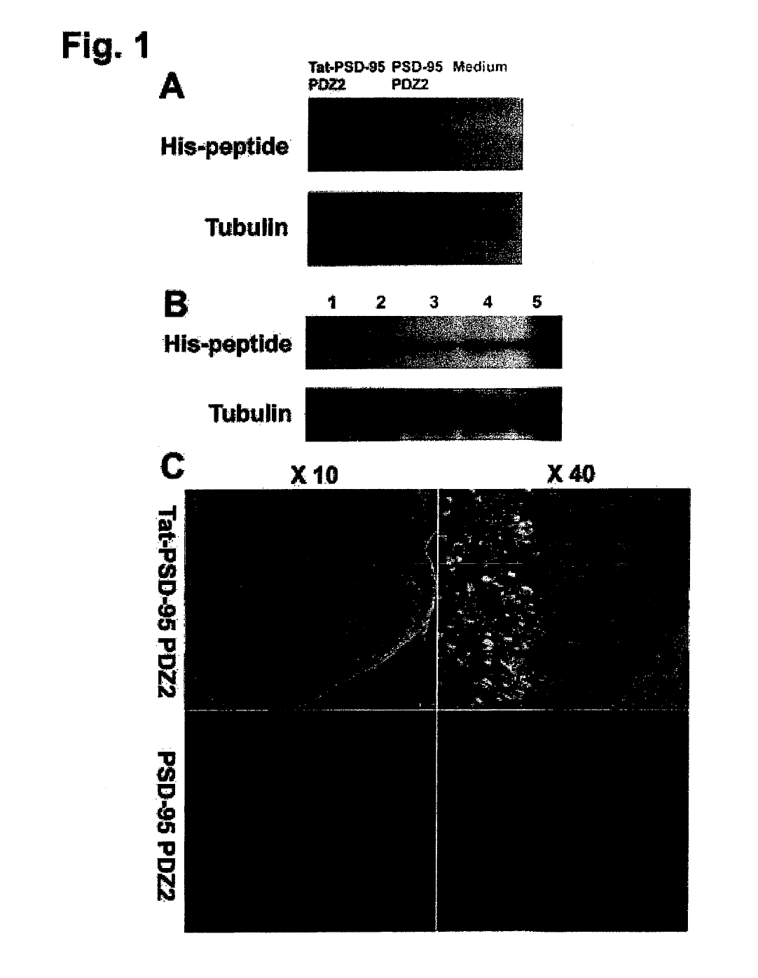

Delivery of Tat Fusion Peptides by PTD. After incubation with Tat-PSD-95 PDZ2 or PSD-95 PDZ2 for 30 min, the cultured spinal neurons were processed to determine whether these peptides containing 6×His were transported into the neurons. Western blotting with anti-His antibody showed that the His-peptide was only detected in the neurons treated with Tat-PSD-95 PDZ2 (FIG. 1A), but not in the neurons treated with PSD-95 PDZ2 or medium alone (FIG. 1A). Furthermore, Western blot analysis and immunohistochemistry were employed to define whether Tat fusion peptide was distributed in the spinal cord after systemic administration. We found that after mice were given intraperitoneally injections of the fusion peptides, Tat-linked fusion peptides (Tat-PSD-95 PDZ2 and mutated Tat-PSD-95 PDZ2), but not PSD-95 PDZ2 without Tat, were delivered into lumbar spinal cord (FIG. 1B). Moreover, Tat-PSD-95 PDZ2 was delivered into the spinal cord in a dose-dependent manner (FIG. 1B). No difference was observed in the PTD-mediated spinal delivery of Tat-PSD-95 PDZ2 and mutated Tat-PSD-95 PDZ2 (FIG. 1B). Immunohistochemical staining also demonstrated that only fusion peptide linked to Tat (Tat-PSD-95 PDZ2) was distributed in the spinal cord after intraperitoneal injection (FIG. 1C).

### Example 3

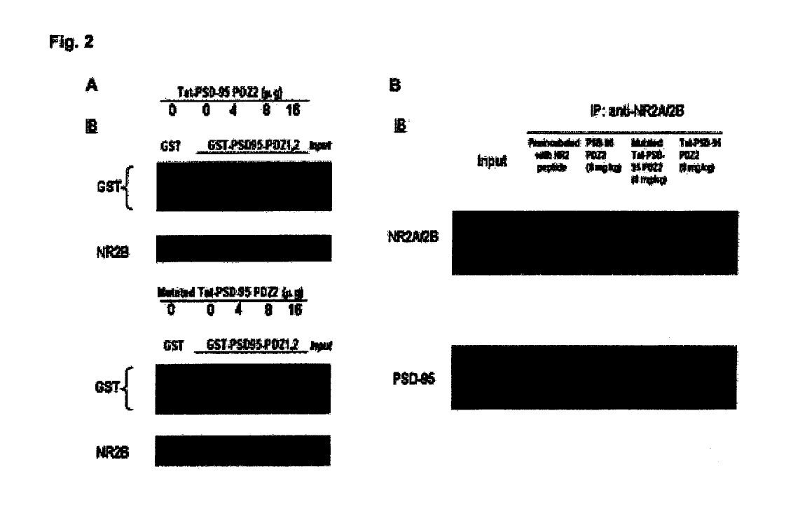

Disruption of NMDAR/PSD-95 Protein Interactions by Tat-PSD-95 PDZ2. GST pull-down and co-immunoprecipitation assays were used to discover whether NMDAR/PSD-95 protein interactions were disrupted by Tat fusion peptides. We found that Tat-PSD-95 PDZ2 markedly disrupted the interactions between NMDAR NR2 subunits and PSD-95 (FIG. 2). However, mutated Tat-PSD-95 PDZ2 had no effect (FIG. 2).

In the GST pull-down assay, both GST-PSD-95 PDZ1,2 and NMDAR subunit NR2B were pulled down by glutathione-agarose (FIG. 2A). Preincubation with Tat-PSD-95 PDZ2 dose-dependently reduced the amount of NR2B, and the treatment with a high dose (16 μg) of Tat-PSD-95 PDZ2 completely prevented NR2B from being pulled down (FIG. 2A). In contrast, preincubation with different doses of mutated Tat-PSD-95 PDZ2 had no effect on the interactions between GST-PSD-95 PDZ1,2 and NR2B, and NR2B was pulled down by glutathione-agarose at a similar level under these conditions (FIG. 2A).

After mice were given intraperitoneal injections of Tat-PSD-95 PDZ2, mutated Tat-PSD-95 PDZ2, or PSD-95 PDZ2 without Tat, NR2A/2B antibody was used to immunoprecipitate NR2A/2B and its interacting proteins from spinal cord homogenates (FIG. 2B). We found that Tat-PSD-95 PDZ2 (8 mg/kg) markedly blocked the interaction between NR2A/2B and PSD-95 but that neither mutated Tat-PSD-95 PDZ2 (8 mg/kg) nor PSD-95 PDZ2 (8 mg/kg) had an effect on this interaction. The specificity of the NR2A/2B antibody was verified by preincubation with NR2 peptide (FIG. 2B).

### Example 4

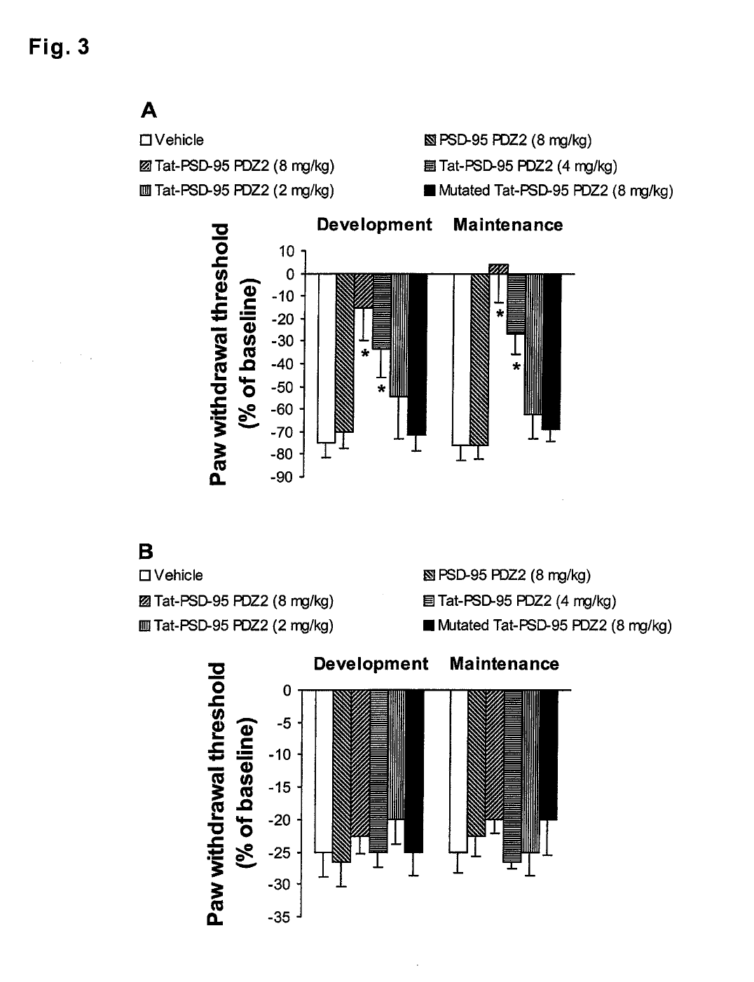

Effect of Tat-PSD-95 PDZ2 on CFA-Induced Inflammatory Pain Behaviors. Both intraperitoneal (systemic) and intrathecal (local) injections were used to assess the effect of Tat-PSD-95 PDZ2 on CFA-induced inflammatory pain behaviors. After being given intraperitoneal injections of Tat-PSD-95 PDZ2 at different doses [2 mg/kg (n=8), 4 mg/kg (n=8), and 8 mg/kg (n=8)], mutated Tat-PSD-95 PDZ2 (8 mg/kg; n=8), or PSD-95 PDZ2 without Tat (8 mg/kg; n=6), mice were tested for paw withdrawal thresholds to examine the effect of these peptides on CFA-induced inflammatory pain behaviors in the development and maintenance phases. We found that Tat-PSD-95 PDZ2 dose-dependently inhibited inflammatory sensitization of the behavioral response induced by CFA injection on the ipsilateral side (FIG. 3A). However, mutated Tat-PSD-95 PDZ2 or PSD-95 PDZ2 without Tat had no effect compared to the vehicle-treated group (n=6). On the contralateral side, paw withdrawal thresholds in these peptides-treated groups were not significantly different from those of the vehicle-treated group. Similarly, intrathecal injection of Tat-PSD-95 PDZ2 at different doses [2.5 μg/5 μl (n=8), 5 μg/5 μl (n=8), and 10 μg/5 μl (n=8)] also dose-dependently inhibited the CFA-induced decrease of paw withdrawal threshold on the ipsilateral side (FIG. 3B), but intrathecal injections of mutated Tat-PSD-95 PDZ2 (10 μg/5 μl; n=8) or PSD-95 PDZ2 without Tat (10 μg/5 μl; n=8) had no effect compared to the vehicle-treated group (n=6). On the contralateral side, intrathecally injected these peptides did not significantly influence paw withdrawal threshold.

The effects of these fusion peptides on the baseline behavior and locomotor function of mice were tested to serve as controls in our experimental design. The mice showed normal grooming behavior and normal levels of activity after intraperitoneal or intrathecal injections of these peptides. Furthermore, none of these peptides had an effect on the baseline paw withdrawal threshold of the mice or on the tests of locomotor function. The baseline paw withdrawal thresholds in these peptides-treated groups were not significantly different from those of the vehicle-treated group. The scores for placing, grasping, and righting reflexes in these peptides-treated groups were also not significantly different from those of the vehicle-treated group.

### Example 5

### CNS Delivery of Tat Peptides after Intraperitoneal Injection

Western blotting showed that after intraperitoneal injection, Tat-linked fusion peptides (Tat-PSD-95 PDZ2 and mutated Tat-PSD-95 PDZ2), but not PSD-95 PDZ2 without Tat, were delivered into cerebral cortex, hippocampus and lumbar spinal cord of the mice (data not shown). Moreover, Tat-P SD-95 PDZ2 was delivered into the spinal cord in a dose-dependent manner (data not shown). No significant difference was observed in the PTD-mediated spinal delivery of Tat-PSD-95 PDZ2 and mutated Tat-PSD-95 PDZ2 (data not shown).

### Example 6

### Tat-PSD-95 PDZ2 Markedly Disrupted the Interactions Between NMDAR NR2 Subunits and PSD-95

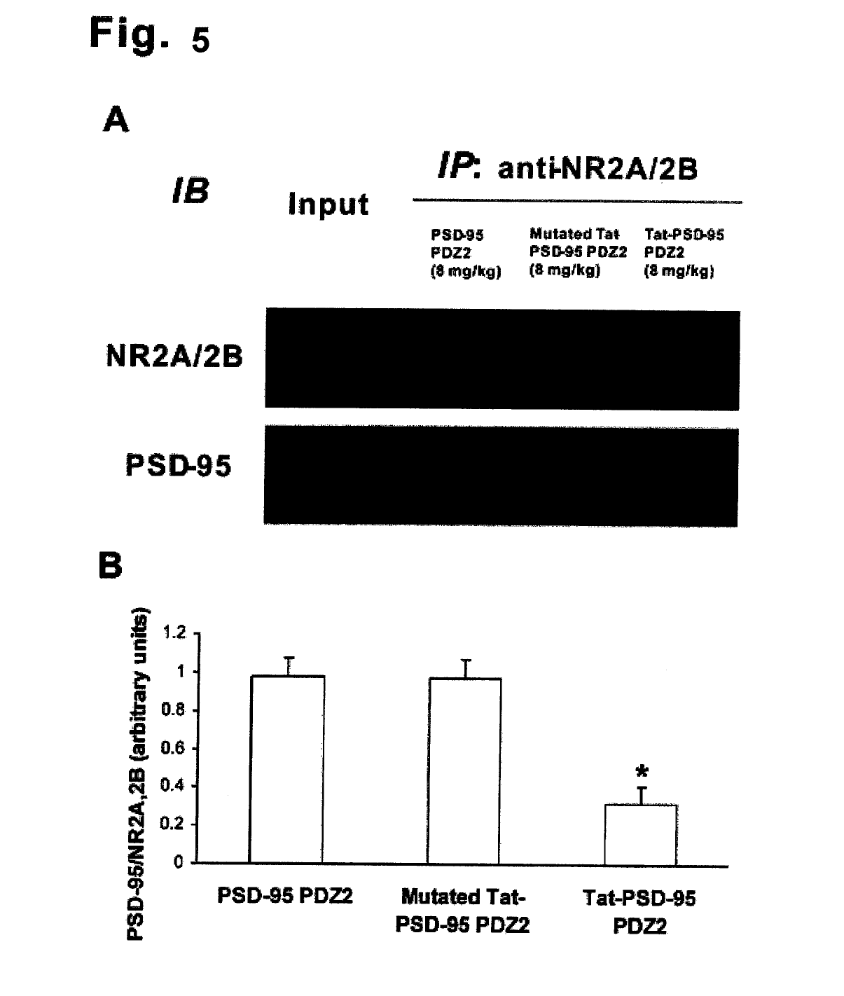

Co-immunoprecipitation assay was used to discover whether NMDAR/PSD-95 protein interactions were interrupted by Tat fusion peptides. We found that Tat-PSD-95 PDZ2 markedly disrupted the interactions between NMDAR NR2 subunits and PSD-95 (FIG. 5). However, mutated Tat-PSD-95 PDZ2 had no effect (FIG. 5).

After mice were given intraperitoneal injection of Tat-PSD-95 PDZ2, mutated Tat-PSD-95 PDZ2, or PSD-95 PDZ2 without Tat, NR2A/2B antibody was used to immunoprecipitate NR2A/2B and its interacting proteins from spinal cord homogenates (FIG. 5). We found that Tat-PSD-95 PDZ2 (8 mg/kg) markedly blocked the interaction between NR2A/2B and PSD-95 but that neither mutated Tat-PSD-95 PDZ2 (8 mg/kg) nor PSD-95 PDZ2 (8 mg/kg) had an effect on this interaction. The specificity of the NR2A/2B antibody was verified by preincubation with NR2 peptide. No bands were detected in this condition (data not shown).

### Example 7

### Effect of Tat Fusion Peptides on the Threshold for Halothane Anesthesia

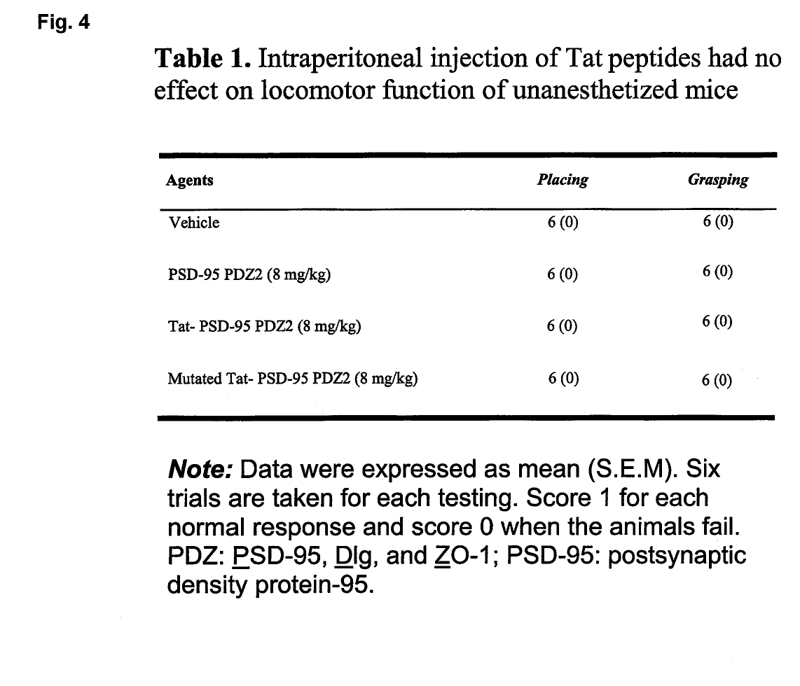

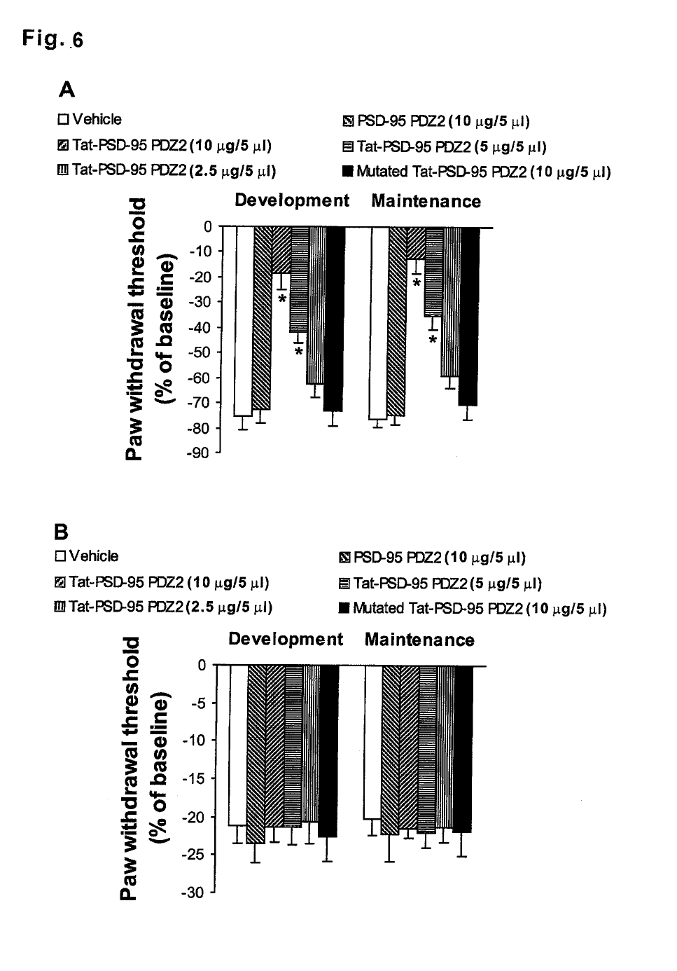

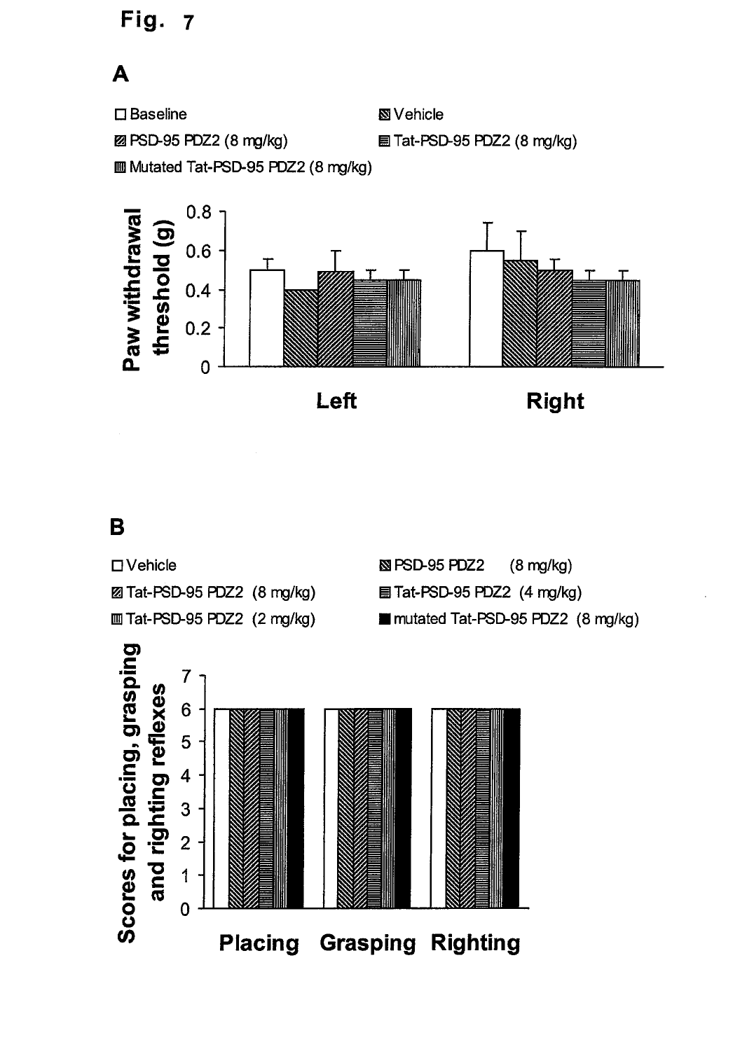

After mice were given intraperitoneal injection of the fusion peptides, halothane MAC and RREC50 were measured respectively. We found that Tat-PSD-95 PDZ2 dose-dependently reduced halothane MAC and RREC50 (FIGS. 6, 7). However, mutated Tat-PSD-95 PDZ2 and PSD-95 PDZ2 without Tat had no effect (FIGS. 6, 7). As a control, we observed that these peptides had no effect on locomotor function of unanesthetized mice (FIG. 4(Table 1)). The mice showed normal grooming behavior, normal levels of activity, and no significant change in either blood pressure or heart rate after intraperitoneal injection of these peptides.

In the MAC study, the value for halothane MAC in vehicle-treated group was 1.12±0.05. In the groups treated with Tat-PSD-95 PDZ2 at the doses of 2, 4, or 8 mg/kg, the halothane MAC values were 1.11±0.05, 0.99±0.05, or 0.77±0.05, respectively (data not shown). One-way analysis of variance showed that halothane MAC was significantly altered after pretreatment with this peptide (p<0.05). The highest dose (8 mg/kg) of Tat-PSD-95 PDZ2 significantly reduced the halothane MAC compared to the vehicle-treated group (p<0.05). In contrast, intraperitoneal injection with the same dose of mutated Tat-PSD-95 PDZ2 (8 mg/kg) or PSD-95 PDZ2 without Tat (8 mg/kg) had no effect on the halothane MAC (p>0.05).

In the RREC50 study, the value for halothane RREC50 in vehicle-treated group was 0.48±0.02. In the groups treated with Tat-PSD-95 PDZ2 at the doses of 2, 4, or 8 mg/kg, the halothane RREC50 values were 0.45±0.03, 0.37±0.03, or 0.18±0.02, respectively (data not shown). One-way analysis of variance showed that halothane RREC50 was significantly altered after pretreatment with this peptide (p<0.05, data not shown). The two higher doses (4 and 8 mg/kg) of Tat-PSD-95 PDZ2 significantly reduced the halothane RREC50 compared to vehicle-treated group (p<0.05, data not shown). In contrast, intraperitoneal injection of mutated Tat-PSD-95 PDZ2 (8 mg/kg) or PSD-95 PDZ2 without Tat (8 mg/kg) had no effect on the halothane RREC50 (p>0.05, data not shown).

### REFERENCES

1. Ren K, Hylden J L, Williams G M, Ruda M A, Dubner R: The effects of a non-competitive NMDA receptor antagonist, MK-801, on behavioral hyperalgesia and dorsal horn neuronal activity in rats with unilateral inflammation. Pain 1992; 50: 331-44
2. Mao J, Price D D, Hayes R L, Lu J, Mayer D J: Differential roles of NMDA and non-NMDA receptor activation in induction and maintenance of thermal hyperalgesia in rats with painful peripheral mononeuropathy. Brain Res. 1992; 598: 271-8
3. Garry M G, Malik S, Yu J, Davis M A, Yang J: Knock down of spinal NMDA receptors reduces NMDA and formalin evoked behaviors in rat. Neuroreport 2000; 11: 49-55
4. Wei F, Wang G D, Kerchner G A, Kim S J, Xu H M, Chen Z F, Zhuo M: Genetic enhancement of inflammatory pain by forebrain NR2B overexpression. Nat. Neurosci. 2001; 4: 164-9
5. Lukatch H S, Kiddoo C E, Maciver M B: Anesthetic-induced burst suppression EEG activity requires glutamate-mediated excitatory synaptic transmission. Cereb. Cortex 2005; 15: 1322-31
6. Nagele P, Metz L B, Crowder C M: Nitrous oxide (N(2)O) requires the N-methyl-D-aspartate receptor for its action in Caenorhabditis elegans. Proc. Natl. Acad. Sci. U.S.A 2004; 101: 8791-6
7. Pittson S, Himmel A M, MacIver M B: Multiple synaptic and membrane sites of anesthetic action in the CAI region of rat hippocampal slices. BMC. Neurosci. 2004; 5: 52
8. Ranft A, Kurz J, Deuringer M, Haseneder R, Dodt H U, Zieglgansberger W, Kochs E, Eder M, Hapfelmeier G: Isoflurane modulates glutamatergic and GABAergic neurotransmission in the amygdala. Eur. J. Neurosci. 2004; 20: 1276-80
9. Irifune M, Takarada T, Shimizu Y, Endo C, Katayama S, Dohi T, Kawahara M: Propofol-induced anesthesia in mice is mediated by gamma-aminobutyric acid-A and excitatory amino acid receptors. Anesth. Analg. 2003; 97: 424-9
10. Stover J F, Sakowitz O W, Kroppenstedt S N, Thomale U W, Kempski O S, Flugge G, Unterberg A W: Differential effects of prolonged isoflurane anesthesia on plasma, extracellular, and CSF glutamate, neuronal activity, 125I-Mk801 NMDA receptor binding, and brain edema in traumatic brain-injured rats. Acta Neurochir. (Wien.) 2004; 146: 819-30
11. Whitehead K J, Rose S, Jenner P: Halothane anesthesia affects NMDA-stimulated cholinergic and GABAergic modulation of striatal dopamine efflux and metabolism in the rat in vivo. Neurochem. Res. 2004; 29: 835-42
12. Christopherson K S, Hillier B J, Lim W A, Bredt D S: PSD-95 assembles a ternary complex with the N-methyl-D-aspartic acid receptor and a bivalent neuronal NO synthase PDZ domain. J. Biol. Chem. 1999; 274: 27467-73
13. Kornau H C, Schenker L T, Kennedy M B, Seeburg P H: Domain interaction between NMDA receptor subunits and the postsynaptic density protein PSD-95. Science 1995; 269: 1737-40
14. Imamura F, Maeda S, Doi T, Fujiyoshi Y: Ligand binding of the second PDZ domain regulates clustering of PSD-95 with the Kv1.4 potassium channel. J. Biol. Chem. 2002; 277: 3640-6
15. Fang M, Tao Y X, He F, Zhang M, Levine C F, Mao P, Tao F, Chou C L, Sadegh-Nasseri S, Johns R A: Synaptic PDZ domain-mediated protein interactions are disrupted by inhalational anesthetics. J. Biol. Chem. 2003; 278: 36669-75
16. Tao Y X, Johns R A: Effect of the deficiency of spinal PSD-95/SAP90 on the minimum alveolar anesthetic concentration of isoflurane in rats. Anesthesiology 2001; 94: 1010-5
17. Becker-Hapak M, McAllister S S, Dowdy S F: TAT-mediated protein transduction into mammalian cells. Methods 2001; 24: 247-56
18. Tao F, Skinner J, Su Q, Johns R A: New role for spinal Stargazin in alpha-amino-3-hydroxy-5-methyl-4-isoxazolepropionic acid receptor-mediated pain sensitization after inflammation. J. Neurosci. Res. 2006; 84: 867-73
19. Tao Y X, Huang Y Z, Mei L, Johns R A: Expression of PSD-95/SAP90 is critical for N-methyl-D-aspartate receptor-mediated thermal hyperalgesia in the spinal cord. Neuroscience 2000; 98: 201-6
20. Fukuda T, Saito S, Sato S, Harukuni I, Toyooka H: Halothane minimum alveolar anesthetic concentration and neuronal nitric oxide synthase activity of the dorsal horn and the locus ceruleus in rats. Anesth. Analg. 1999; 89: 1035-9
21. Engelhardt T, Lowe P R, Galley H F, Webster N R: Inhibition of neuronal nitric oxide synthase reduces isoflurane MAC and motor activity even in nNOS knockout mice. Br. J. Anaesth. 2006; 96: 361-6
22. Ichinose F, Huang P L, Zapol W M: Effects of targeted neuronal nitric oxide synthase gene disruption and nitroG-L-arginine methylester on the threshold for isoflurane anesthesia. Anesthesiology 1995; 83: 101-8
23. Tao F, Tao Y X, Mao P, Johns R A: Role of postsynaptic density protein-95 in the maintenance of peripheral nerve injury-induced neuropathic pain in rats. Neuroscience 2003; 117: 731-9
24. Denicourt C, Dowdy S F: Protein transduction technology offers novel therapeutic approach for brain ischemia. Trends Pharmacol. Sci. 2003; 24: 216-8
25. Wadia J S, Dowdy S F: Protein transduction technology. Curr. Opin. Biotechnol. 2002; 13: 52-6
26. Aarts M, Liu Y, Liu L, Besshoh S, Arundine M, Gurd J W, Wang Y T, Salter M W and Tymianski M (2002) Treatment of ischemic brain damage by perturbing NMDA receptor-PSD-95 protein interactions. Science 298:846-850.
27. Becker-Hapak M, McAllister S S and Dowdy S F (2001) TAT-mediated protein transduction into mammalian cells. Methods 24:247-256.
28. Bertolini A, Ottani A and Sandrini M (2002) Selective COX-2 inhibitors and dual acting anti-inflammatory drugs: critical remarks. Curr Med Chem 9:1033-1043.
29. Bliss T V and Collingridge G L (1993) A synaptic model of memory: long-term potentiation in the hippocampus. Nature 361:31-39.
30. Boyce S, Wyatt A, Webb J K, O'Donnell R, Mason G, Rigby M, Sirinathsinghji D, Hill R G and Rupniak N M (1999) Selective NMDA NR2B antagonists induce antinociception without motor dysfunction: correlation with restricted localisation of NR2B subunit in dorsal horn. Neuropharmacology 38:611-623.
31. Brenman J E, Chao D S, Gee S H, McGee A W, Craven S E, Santillano D R, Wu Z, Huang F, Xia H, Peters M F, Froehner S C and Bredt D S (1996a) Interaction of nitric oxide synthase with the postsynaptic density protein PSD-95 and alpha1-syntrophin mediated by PDZ domains. Cell 84:757-767.
32. Brenman J E, Christopherson K S, Craven S E, McGee A W and Bredt D S (1996b) Cloning and characterization of postsynaptic density 93, a nitric oxide synthase interacting protein. J Neurosci 16:7407-7415.
33. Cao G, Pei W, Ge H, Liang Q, Luo Y, Sharp F R, Lu A, Ran R, Graham S H and Chen J (2002) In Vivo Delivery of a Bcl-xL Fusion Protein Containing the TAT Protein Transduction Domain Protects against Ischemic Brain Injury and Neuronal Apoptosis. J Neurosci 22:5423-5431.
34. Cho K O, Hunt C A and Kennedy M B (1992) The rat brain postsynaptic density fraction contains a homolog of the Drosophila discs-large tumor suppressor protein. Neuron 9:929-942.
35. Christopherson K S, Hillier B J, Lim W A and Bredt D S (1999) PSD-95 assembles a ternary complex with the N-methyl-D-aspartic acid receptor and a bivalent neuronal NO synthase PDZ domain. J Biol Chem 274:27467-27473.
36. Coderre T J and Van E, I (1994) The utility of excitatory amino acid (EAA) antagonists as analgesic agents. I. Comparison of the antinociceptive activity of various classes of EAA antagonists in mechanical, thermal and chemical nociceptive tests. Pain 59:345-352.
37. Denicourt C and Dowdy S F (2003) Protein transduction technology offers novel therapeutic approach for brain ischemia. Trends Pharmacol Sci 24:216-218.
38. Dworkin R H, Backonja M, Rowbotham M C, Allen R R, Argoff C R, Bennett G J, Bushnell M C, Farrar J T, Galer B S, Haythornthwaite J A, Hewitt D J, Loeser J D, Max M B, Saltarelli M, Schmader K E, Stein C, Thompson D, Turk D C, Wallace M S, Watkins L R and Weinstein S M (2003) Advances in neuropathic pain: diagnosis, mechanisms, and treatment recommendations. Arch Neurol 60:1524-1534.
39. Eisenberg E, McNicol E D and Carr D B (2005) Efficacy and safety of opioid agonists in the treatment of neuropathic pain of nonmalignant origin: systematic review and meta-analysis of randomized controlled trials. JAMA 293:3043-3052.

40. Eisenberg E, McNicol E D and Can D B (2006) Efficacy of mu-opioid agonists in the treatment of evoked neuropathic pain: Systematic review of randomized controlled trials. Eur J Pain 10:667-676.
41. Fang M, Tao Y X, He F, Zhang M, Levine C F, Mao P, Tao F, Chou C L, Sadegh-Nasseri S and Johns R A (2003) Synaptic PDZ domain-mediated protein interactions are disrupted by inhalational anesthetics. J Biol Chem 278:36669-36675.
42. Feldmann M (2002) Development of anti-TNF therapy for rheumatoid arthritis. Nat Rev Immunol 2:364-371.
43. Garry E M, Moss A, Delaney A, O'Neill F, Blakemore J, Bowen J, Husi H, Mitchell R, Grant S G and Fleetwood-Walker S M (2003) Neuropathic sensitization of behavioral reflexes and spinal NMDA receptor/CaM kinase II interactions are disrupted in PSD-95 mutant mice. Curr Biol 13:321-328.
44. Garry M G, Malik S, Yu J, Davis M A and Yang J (2000) Knock down of spinal NMDA receptors reduces NMDA and formalin evoked behaviors in rat. Neuroreport 11:49-55.
45. Hansson P T and Dickenson A H (2005) Pharmacological treatment of peripheral neuropathic pain conditions based on shared commonalities despite multiple etiologies. Pain 113:251-254.
46. Hylden J L and Wilcox G L (1980) Intrathecal morphine in mice: a new technique. Eur J Pharmacol 67:313-316.
47. Jaffrey S R and Snyder S H (1995) Nitric oxide: a neural messenger. Annu Rev Cell Dev Biol 11:417-440.
48. Kornau H C, Schenker L T, Kennedy M B and Seeburg P H (1995) Domain interaction between NMDA receptor subunits and the postsynaptic density protein PSD-95. Science 269:1737-1740.
49. Laird J M, Herrero J F, Garcia de la R P and Cervero F (1997) Analgesic activity of the novel COX-2 preferring NSAID, meloxicam in mono-arthritic rats: central and peripheral components. Inflamm Res 46:203-210.
50. Luque J M, Bleuel Z, Malherbe P and Richards J G (1994) Alternatively spliced isoforms of the N-methyl-D-aspartate receptor subunit 1 are differentially distributed within the rat spinal cord. Neuroscience 63:629-635.
51. Mao J, Price D D, Hayes R L, Lu J and Mayer D J (1992) Differential roles of NMDA and non-NMDA receptor activation in induction and maintenance of thermal hyperalgesia in rats with painful peripheral mononeuropathy. Brain Res 598:271-278.
52. Moon I S, Apperson M L and Kennedy M B (1994) The major tyrosine-phosphorylated protein in the postsynaptic density fraction is N-methyl-D-aspartate receptor subunit 2B. Proc Natl Acad Sci USA 91:3954-3958.
53. Nelson R J, Demas G E, Huang P L, Fishman M C, Dawson V L, Dawson T M and Snyder S H (1995) Behavioural abnormalities in male mice lacking neuronal nitric oxide synthase. Nature 378:383-386.
54. O'Brien R J, Mammen A L, Blackshaw S, Ehlers M D, Rothstein J D and Huganir R L (1997) The development of excitatory synapses in cultured spinal neurons. J Neurosci 17:7339-7350.
55. Reimold A M (2003) New indications for treatment of chronic inflammation by TNF-alpha blockade. Am J Med Sci 325:75-92.
56. Ren K, Hylden J L, Williams G M, Ruda M A and Dubner R (1992) The effects of a non-competitive NMDA receptor antagonist, MK-801, on behavioral hyperalgesia and dorsal horn neuronal activity in rats with unilateral inflammation. Pain 50:331-344.
57. Sheng M (2001) Molecular organization of the postsynaptic specialization. Proc Natl Acad Sci USA 98:7058-7061.
58. Tao F, Tao Y X, Gonzalez J A, Fang M, Mao P and Johns R A (2001) Knockdown of PSD-95/SAP90 delays the development of neuropathic pain in rats. Neuroreport 12:3251-3255.
59. Tao F, Tao Y X, Mao P and Johns R A (2003a) Role of postsynaptic density protein-95 in the maintenance of peripheral nerve injury-induced neuropathic pain in rats. Neuroscience 117:731-739.
60. Tao F, Tao Y X, Mao P, Zhao C, Li D, Liaw W J, Raja S N and Johns R A (2003b) Intact carrageenan-induced thermal hyperalgesia in mice lacking inducible nitric oxide synthase. Neuroscience 120:847-854.
61. Tao F, Tao Y X, Zhao C, Dore S, Liaw W J, Raja S N and Johns R A (2004) Differential roles of neuronal and endothelial nitric oxide synthases during carrageenan-induced inflammatory hyperalgesia. Neuroscience 128:421-430.
62. Tao Y X, Huang Y Z, Mei L and Johns R A (2000) Expression of PSD-95/SAP90 is critical for N-methyl-D-aspartate receptor-mediated thermal hyperalgesia in the spinal cord. Neuroscience 98:201-206.
63. Wadia J S and Dowdy S F (2002) Protein transduction technology. Curr Opin Biotechnol 13:52-56.
64. Wei F, Wang G D, Kerchner G A, Kim S J, Xu H M, Chen Z F and Zhuo M (2001) Genetic enhancement of inflammatory pain by forebrain NR2B overexpression. Nat Neurosci 4:164-169.
65. Yang D and Gereau R W (2003) Peripheral group II metabotropic glutamate receptors mediate endogenous anti-allodynia in inflammation. Pain 106:411-417.

1. A method for relieving acute or chronic pain in a human, comprising:
administering to a subject in need thereof an effective amount of a fusion protein which comprises a cell membrane transduction domain of HIV1 Tat and a PDZ domain of a protein selected from the group consisting of PICK1, PSD93 and PSD95, whereby acute or chronic pain experienced by the subject is relieved.

2. The method of claim 1 wherein the fusion protein comprises a PDZ2 domain of PSD93.

3. The method of claim 1 wherein the fusion protein comprises a PDZ2 domain of PSD95.

4. The method of claim 1 wherein the fusion protein is administered intraperitoneally.

5. The method of claim 1 wherein the fusion protein is administered systemically or intrathecally.

6. A method for treating or preventing allodynia or hyperalgesia in a human, comprising:
administering to a subject in need thereof an effective amount of a fusion protein which comprises a cell membrane transduction domain of HIV1 Tat and a PDZ domain of a protein selected from the group consisting of PICK1, PSD93 and PSD95, whereby allodynia or hyperalgesia experienced by the subject is relieved.

7. The method of claim 6 wherein the fusion protein comprises a PDZ2 domain of PSD93.

8. The method of claim 6 wherein the fusion protein comprises a PDZ2 domain of PSD95.

9. The method of claim 6 wherein the fusion protein is administered intraperitoneally.

10. The method of claim 6 wherein the fusion protein is administered systemically or intrathecally.

11. A method of reducing a threshold for anesthesia in a human, comprising:
administering to a subject an anesthetic and a fusion protein which comprises a cell membrane transduction domain of HIV1 Tat and a PDZ domain of a protein selected from the group consisting of MUPP1, PSD93 and PSD95, wherein the amount of anesthetic administered is less than the amount required in the absence of the agent to achieve a desired anesthetic effect, whereby the desired anesthetic effect is achieved.

12. The method of claim 11 wherein the fusion protein comprises a PDZ2 domain of PSD93.

13. The method of claim 11 wherein the fusion protein comprises a PDZ2 domain of PSD95.

14. The method of claim 11 wherein the agent is administered intraperitoneally.

15. The method of claim 11 wherein the agent is administered systemically or intrathecally.

16. The method of claim 11 wherein the anesthetic is selected from the group consisting of halothane, isoflurane, desflurane, xenon, and sevoflurane.

17. The method of claim 11 wherein the anesthetic is an inhalational anesthetic.

18. An isolated and purified fusion protein which comprises a cell membrane transduction domain of HIV1 Tat and a PDZ domain of a protein selected from the group consisting of PICK1, MUPP1, PSD95 and PSD93.

19. The isolated and purified fusion protein of claim 18 wherein the fusion protein comprises a PDZ2 domain of PSD93.

20. The isolated and purified fusion protein of claim 18 wherein the fusion protein comprises a PDZ2 domain of PSD95.

21. The isolated and purified fusion protein of claim 18 wherein the fusion protein is administered intraperitoneally.

22. The isolated and purified fusion protein of claim 18 wherein the fusion protein is administered systemically or intrathecally.

23. The isolated and purified fusion protein of claim 18 wherein the fusion protein comprises the PDZ domain of PICK1.

24. The isolated and purified fusion protein of claim 18 wherein the fusion protein comprises PDZ13 of MUPP1.

25. A method of anesthetizing or sedating a human, comprising:
administering to a subject a fusion protein which comprises a cell membrane transduction domain of HIV1 Tat and a PDZ domain of a protein selected from the group consisting of MUPP1, PSD93 and PSD95, whereby the agent renders the subject unconscious or sedated.

26. The method of claim 25 wherein the fusion protein comprises a PDZ2 domain of PSD93.

27. The method of claim 25 wherein the fusion protein comprises a PDZ2 domain of PSD95.

28. The method of claim 25 wherein the fusion protein is administered intraperitoneally.

29. The method of claim 25 wherein the fusion protein is administered systemically or intrathecally.

30. The method of claim 1, 6, 11, or 25 wherein the fusion protein comprises PDZ1 domain of PSD95.

31. The method of claim 1, 6, 11, or 25 wherein the fusion protein comprises PDZ3 domain of PSD95.

32. The method of claim 1, 6, 11, or 25 wherein the fusion protein comprises PDZ1 domain of PSD93.

33. The method of claim 1, 6, 11, or 25 wherein the fusion protein comprises PDZ3 domain of PSD93.

34. The method of claim 1, 6, 11, or 25 wherein at least two fusion proteins are administered comprising different PDZ domains.

35. A composition comprising at least two isolated and purified fusion proteins which each comprise a cell membrane transduction domain of HIV1 Tat and a PDZ domain of a protein selected from the group consisting of PICK1, MUPP1, PSD95 and PSD93.

36. The composition of claim 35 comprising at least three fusion proteins.

37. The composition of claim 35 wherein the PDZ domains are of PSD95.

38. The composition of claim 35 wherein the PDZ domains are of PSD93.

39. The composition of claim 35 wherein at least one of the fusion proteins comprises a PDZ2 domain.


SEQUENCE LISTING (13 sequences)

SEQ ID NO: 1 - protein, 870 aa, Homo sapiens
MFFACYCALRTNVKKYRYQDEDAPHDHSLPRLTHEVRGPELVHVSEKNLSQIENVHGYVLQSHISPLKASPAPIIVNTDTLDTIPYVNGTEIEYEFEEITLERGNSGLGFSIAGGTDNPHIGDDPGIFITKIIPGGAAAEDGRLRVNDCILRVNEVDVSEVSHSKAVEALKEAGSIVRLYVRRRRPILETVVEIKLFKGPKGLGFSIAGGVGNQHIPGDNSIYVTKIIDGGAAQKDGRLQVGDRLLMVNNYSLEEVTHEEAVAILKNTSEVVYLKVGKPTTIYMTDPYGPPDITHSYSPPMENHLLSGNNGTLEYKTSLPPISPGRYSPIPKHMLVDDDYTRPPEPVYSTVNKLCDKPASPRHYSPVECDKSFLLSAPYSHYHLGLLPDSEMTSHSQHSTATRQPSMTLQRAVSLEGEPRKVVLHKGSTGLGFNIVGGEDGEGIFVSFILAGGPADLSGELQRGDQILSVNGIDLRGASHEQAAAALKGAGQTVTIIAQYQPEDYARFEAKIHDLREQMMNHSMSSGSGSLRTNQKRSLYVRAMFDYDKSKDSGLPSQGLSFKYGDILHVINASDDEWWQARRVMLEGDSEEMGVIPSKRRVERKERARLKTVKFNAKPGVIDSKGSFNDKRKKSFIFSRKFPFYKNKEQSEQETSDPERGQEDLILSYEPVTRQEINYTRPVIILGPMKDRINDDLISEFPDKFGSCVPHTTRPKRDYEVDGRDYHFVISREQMEKDIQEHKFIEAGQYNDNLYGTSVQSVRFVAERGKHCILDVSGNAIKRLQVAQLYPIAIFIKPRSLEPLMEMNKRLTEEQAKKTYDRAIKLEQEFGEYFTAIVQGDTLEDIYNQCKLVIEEQSGPFIWIPSKEKL

SEQ ID NO: 2 - protein, 77 aa, Homo sapiens
SGLGFSIAGGTDNPHIGDDPGIFITKIIPGGAAAEDGRLRVNDCILRVNEVDVSEVSHSKAVEALKEAGSIVRLYVR

SEQ ID NO: 3 - protein, 86 aa, Homo sapiens
VVEIKLFKGPKGLGFSIAGGVGNQHIPGDNSIYVTKIIDGGAAQKDGRLQVGDRLLMVNNYSLEEVTHEEAVAILKNTSEVVYLKV

SEQ ID NO: 4 - protein, 81 aa, Homo sapiens
PRKVVLHKGSTGLGFNIVGGEDGEGIFVSFILAGGPADLSGELQRGDQILSVNGIDLRGASHEQAAAALKGAGQTVTIIAQ

SEQ ID NO: 5 - protein, 767 aa, Homo sapiens
MSQRPRAPRSALWLLAPPLLRWAPPLLTVLHSDLFQALLDILDYYEASLSESQKYRYQDEDTPPLEHSPAHLPNQANSPPVIVNTDTLEAPGYELQVNGTEGEMEYEEITLERGNSGLGFSIAGGTDNPHIGDDPSIFITKIIPGGAAAQDGRLRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITTSYSQHLDNEISHSSYLGTDYPTAMTPTSPRRYSPVAKDLLGEEDIPREPRRIVIHRGSTGLGFNIVGGEDGEGIFISFILAGGPADLSGELRKGDQILSVNGVDLRNASHEQAAIALKNAGQTVTIIAQYKPEEYSRFEAKIHDLREQLMNSSLGSGTASLRSNPKRGFYIRALFDYDKTKDCGFLSQALSFRFGDVLHVIDASDEEWWQARRVHSDSETDDIGFIPSKRRVERREWSRLKAKDWGSSSGSQGREDSVLSYETVTQMEVHYARPIIILGPTKDRANDDLLSEFPDKFGSCVPHTTRPKREYEIDGRDYHFVSSREKMEKDIQAHKFIEAGQYNSHLYGTSVQSVREVAEQGKHCILDVSANAVRRLQAAHLHPIAIFIRPRSLENVLEINKRITEEQARKAFDRATKLEQEFTECFSAIVEGDSFEEIYHKVKRVIEDLSGPYIWVPARERL

SEQ ID NO: 6 - protein, 84 aa, Homo sapiens
EITLERGNSGLGFSIAGGTDNPHIGDDPSIFITKIIPGGAAAQDGRLRVNDSILFVNEVDVREVTHSAAVEALKEAGSIVRLYV

SEQ ID NO: 7 - protein, 90 aa, Homo sapiens
VMEIKLIKGPKGLGFSIAGGVGNQHIPGDNSIYVTKIIEGGAAHKDGRLQIGDKILAVNSVGLEDVMHEDAVAALKNTYDVVYLKVAKPS

SEQ ID NO: 8 - protein, 81 aa, Homo sapiens
PRRIVIHRGSTGLGFNIVGGEDGEGIFISFILAGGPADLSGELRKGDQILSVNGVDLRNASHEQAAIALKNAGQTVTIIAQ

SEQ ID NO: 9 - protein, 11 aa, Homo sapiens
YGRKKRRQRRR

SEQ ID NO: 10 - protein, 415 aa, Homo sapiens
MFADLDYDIEEDKLGIPTVPGKVTLQKDAQNLIGISIGGGAQYCPCLYIVQVFDNTPAALDGTVAAGDEITGVNGRSIKGKTKVEVAKMIQEVKGEVTIHYNKLQADPKQGMSLDIVLKKVKHRLVENMSSGTADALGLSRAILCNDGLVKRLEELERTAELYKGMTEHTKNLLRAFYELSQTHRAFGDVFSVIGVREPQPAASEAFVKFADAHRSIEKFGIRLLKTIKPMLTDLNTYLNKAIPDTRLTIKKYLDVKFEYLSYCLKVKEMDDEEYSCIALGEPLYRVSTGNYEYRLILRCRQEARARFSQMRKDVLEKMELLDQKHVQDIVFQLQRLVSTMSKYYNDCYAVLRDADVFPIEVDLAHTTLAYGLNQEEFTDGEEEEEEEDTAAGEPSRDTRGAAGPLDKGGSWCDS

SEQ ID NO: 11 - protein, 77 aa, Homo sapiens
VTLQKDAQNLIGISIGGGAQYCPCLYIVQVFDNTPAALDGTVAAGDEITGVNGRSIKGKTKVEVAKMIQEVKGEVTI

SEQ ID NO: 12 - protein, 2042 aa, Homo sapiens
MLEAIDKNRALHAAERLQTKLRERGDVANEDKLSLLKSVLQSPLFSQILSLQTSVQQLKDQVNIATSATSNIEYAHVPHLSPAVIPTLQNESFLLSPNNGNLEALTGPGIPHINGKPACDEFDQLIKNMAQGRHVEVFELLKPPSGGLGFSVVGLRSENRGELGIFVQEIQEGSVAHRDGRLKETDQILAINGQALDQTITHQQAISILQKAKDTVQLVIARGSLPQLVSPIVSRSPSAASTISAHSNPVHWQHMETIELVNDGSGLGFGIIGGKATGVIVKTILPGGVADQHGRLCSGDHILKIGDTDLAGMSSEQVAQVLRQCGNRVKLMIARGAIEERTAPTALGITLSSSPTSTPELRVDASTQKGEESETFDVELTKNVQGLGITIAGYIGDKKLEPSGIFVKSITKSSAVEHDGRIQIGDQIIAVDGTNLQGFTNQQAVEVLRHTGQTVLLTLMRRGMKQEAELMSREDVTKDADLSPVNASIIKENYEKDEDFLSSTRNTNILPTEEEGYPLLSAEIEEIEDAQKQEAALLTKWQRIMGINYEIVVAHVSKFSENSGLGISLEATVGHHFIRSVLPEGPVGHSGKLFSGDELLEVNGITLLGENHQDVVNILKELPIEVTMVCCRRTVPPTTQSELDSLDLCDIELTEKPHVDLGEFIGSSETEDPVLAMTDAGQSTEEVQAPLAMWEAGIQHIELEKGSKGLGFSILDYQDPIDPASTVIIIRSLVPGGIAEKDGRLLPGDRLMFVNDVNLENSSLEEAVEALKGAPSGTVRIGVAKPLPLSPEEGYVSAKEDSFLYPPHSCEEAGLADKPLFRADLALVGTNDADLVDESTFESPYSPENDSIYSTQASILSLHGSSCGDGLNYGSSLPSSPPKDVIENSCDPVLDLHMSLEELYTQNLLQRQDENTPSVDISMGPASGFTINDYTPANAIEQQYECENTIVWTESHLPSEVISSAELPSVLPDSAGKGSEYLLEQSSLACNAECVMLQNVSKESFERTINIAKGNSSLGMTVSANKDGLGMIVRSIIHGGAISRDGRIAIGDCILSINEESTISVTNAQARAMLRRHSLIGPDIKITYVPAEHLEEFKISLGQQSGRVMALDIFSSYTGRDIPELPEREEGEGEESELQNTAYSNWNQPRRVELWREPSKSLGISIVGGRGMGSRLSNGEVMRGIFIKHVLEDSPAGKNGTLKPGDRIVEVDGMDLRDASHEQAVEAIRKAGNPVVFMVQSIINRPRKSPLPSLLHNLYPKYNFSSTNPFADSLQINADKAPSQSESEPEKAPLCSVPPPPPSAFAEMGSDHTQSSASKISQDVDKEDEFGYSWKNIRERYGTLTGELHMIELEKGHSGLGLSLAGNKDRSRMSVFIVGIDPNGAAGKDGRLQIADELLEINGQILYGRSHQNASSIIKCAPSKVKIIFIRNKDAVNQMAVCPGNAVEPLPSNSENLQNKETEPTVTTSDAAVDLSSFKNVQHLELPKDQGGLGIAISEEDTLSGVIIKSLTEHGVAATDGRLKVGDQILAVDDEIVVGYPIEKFISLLKTAKMTVKLTIHAENPDSQAVPSAAGAASGEKKNSSQSLMVPQSGSPEPESIRNTSRSSTPAIFASDPATCPIIPGCETTIEISKGRTGLGLSIVGGSDTLLGAIIIHEVYEEGAACKDGRLWAGDQILEVNGIDLRKATHDEAINVLRQTPQRVRLTLYRDEAPYKEEEVCDTLTIELQKKPGKGLGLSIVGKRNDTGVFVSDIVKGGIADADGRLMQGDQILMVNGEDVRNATQEAVAALLKCSLGTVTLEVGRIKAGSSTSESLESSSKKNALASEIQGLRTVEMKKGPTDSLGISIAGGVGSPLGDVPIFIAMMHPTGVAAQTQKLRVGDRIVTICGTSTEGMTHTQAVNLLKNASGSIEMQVVAGGDVSVVTGHQQEPASSSLSFTGLTSSSIFQDDLGPPQCKSITLERGPDGLGFSIVGGYGSPHGDLPIYVKTVFAKGAASEDGRLKRGDQIIAVNGQSLEGVTHEEAVAILKRTKGTVTLMVLS

SEQ ID NO: 13 - protein, 84 aa, Homo sapiens
KSITLERGPDGLGFSIVGGYGSPHGDLPIYVKTVFAKGAASEDGRLKRGDQIIAVNGQSLEGVTHEEAVAILKRTKGTVTLMVL